(19) United States
(12) Patent Application Publication
(10) Pub. No.: US 2022/0301654 A1
(43) Pub. Date: Sep. 22, 2022

(54) SYSTEMS AND METHODS FOR PREDICTING AND MONITORING TREATMENT RESPONSE FROM CELL-FREE NUCLEIC ACIDS

(71) Applicant: Grail, LLC, Menlo Park, CA (US)

(72) Inventors: Jing XIANG, San Carlos, CA (US); Anton VALOUEV, Palo Alto, CA (US); David BURKHARDT, San Francisco, CA (US); Nathan HUNKAPILLER, Belmont, CA (US); Eric FUNG, Los Altos, CA (US); Xiaoji CHEN, Newark, CA (US); Byoungsok JUNG, Atherton, CA (US)

(21) Appl. No.: 17/638,904

(22) PCT Filed: Aug. 28, 2020
(86) PCT No.: PCT/US2020/048612

§ 371 Date: Feb. 28, 2022

Related U.S. Application Data
(60) Provisional application No. 62/893,119, filed on Aug. 28, 2019.

(51) Int. Cl. G16B 20/20; G16B 40/20; C12Q 1/6886; G16H 10/40; G16H 50/20; G16H 20/10

(52) U.S. Cl. CPC G16B 20/20; G16B 40/20; C12Q 1/6886; G16H 10/40; G16H 50/20; G16H 20/10; C12Q 1/6869

(57) ABSTRACT

Methods and systems for determining a subject's likelihood of responding to a treatment by assessing the subject's cell-free DNA (cfDNA) sample include receiving sequence data gathered from sequencing the cfDNA sample, generating a feature matrix of values that correspond to synonymous and nonsynonymous mutations detected in the sequence data, and predicting, based on analysis of the feature matrix at a TMB prediction model, a tumor mutational burden (TMB) for a tissue of interest at the subject. The predicted TMB is evaluated to determine whether a set of criteria indicating a likely response to treatment is met. The set of criteria can include criterion(s) that are met when the predicted TMB is high, when the predicted TMB corresponds to a predicted tumoral heterogeneity indicative of homogeneous tissue, when the predicted TMB corresponds to a tumor fraction indicative of a positive responder, or any combination thereof.

### CROSS-REFERENCE

This application claims the benefit of U.S. Provisional Application No. 62/893,119, filed Aug. 28, 2019, and entitled “Systems and Methods for Predicting Treatment Response from Cell-Free Nucleic Acids,” the application of which is incorporated herein by reference in its entirety.

### BACKGROUND OF THE INVENTION

Some cancer patients respond to treatments, such as immunotherapy. Prediction and monitoring of patient responsiveness to such treatments can lead to better treatment, and thus, lower mortality associated with cancers. Accordingly, there is a need in the art for improved methods for predicting and monitoring of treatment response.

### BRIEF SUMMARY OF THE INVENTION

This disclosure generally relates to evaluating treatment response, and more particularly, to predicting, monitoring, or otherwise determining treatment response based on analysis of cell-free nucleic acids (cfNAs).

In some aspects, a method is provided for determining a subject's likelihood of responding to a treatment by assessing a cell-free DNA (cfDNA) sample collected from the subject. The method includes receiving sequence data gathered from sequencing the cfDNA sample, generating a feature matrix comprising feature values corresponding to synonymous and nonsynonymous mutations in the sequence data, and predicting a tumor mutational burden (TMB) for a tissue of interest at the subject using a TMB prediction model that receives the feature matrix as input and outputs a predicted TMB. The method includes, subsequent to determining the predicted TMB, determining whether a set of criteria has been met, whereby the set of criteria includes at least one criterion that is met when the predicted TMB is high. The method includes, in accordance with a determination that the set of criteria has been met, determining that the subject is likely to respond to the treatment, and in accordance with a determination that the set of criteria has not been met, determining that the subject is not likely to respond to the treatment.

Various embodiments are contemplated in the present invention. In some embodiments, the predicted TMB is determined to be high when the predicted TMB exceeds a predetermined value.

In some embodiments, the feature values include one or more of: a number of nonsynonymous somatic mutations for each region of a plurality of regions included in an assay used to sequence the cfDNA sample, a total number of somatic mutations in the cfDNA sample, and a total number of nonsynonymous somatic mutations in the cfDNA sample. Further, in some embodiments, the assay includes a plurality of genomic regions and each region comprises an individual gene.

In some embodiments, the predicted TMB represents an estimated total number of nonsynonymous somatic mutations for the tissue of interest at the subject.

In some embodiments, the treatment comprises an immunotherapy treatment. Further, in some embodiments, the immunotherapy treatment comprises an immuno oncology treatment.

In some embodiments, the method includes, in accordance with the determination that the subject is likely to respond to the treatment, continuing administration of the treatment to the subject, and in accordance with the determination that the subject is not likely to respond to the treatment, altering administration of the treatment to the subject.

In some embodiments, the TMB prediction model comprises a statistical model trained with a training set comprising training data obtained from sequencing a plurality of training samples of cfDNA collected from a plurality of subjects, wherein the training data obtained from each training sample corresponds to matched tissue data obtained from a tumoral tissue sample collected from the same subject. Further, in some embodiments, the training data is obtained from targeted sequencing of the plurality of training samples. In some embodiments, the matched tissue data is obtained from whole exome sequencing of the tumoral tissue sample.

In some embodiments, the method includes, for each training sample in the plurality of training samples: labeling the training data with a corresponding ground truth TMB determined from the corresponding matched tissue data, generating a predicted TMB from the labeled training data using the statistical model, and correlating the predicted TMB with the corresponding ground truth TMB. In some embodiments, the statistical model comprises a L1 penalized linear regression model. In some embodiments, each train sample corresponds to a cancer stage III or stage IV condition. Further, in some embodiments, each training sample of cfDNA has a tumor fraction that exceeds a minimum tumor fraction. In some embodiments, the tumor fraction comprises a maximum allele frequency of all mutations in the training sample.

In some embodiments, the set of criteria includes a criterion that is met when the predicted TMB is high and corresponds to a predicted tumoral heterogeneity (TH) that is indicative of a homogeneous tissue.

In some embodiments, the method includes, subsequent to the determination that the predicted TMB is high, predicting, based on the sequence data, the TH for the tissue of interest at the subject; determining whether the predicted TH is indicative of homogeneous or heterogeneous tissue; in accordance with a determination that the predicted TH is indicative of the homogeneous tissue, determining that the subject is likely to respond to the treatment; and in accordance with a determination that the predicted TH is indicative of the heterogeneous tissue, determining that the subject is not likely to respond to the treatment.

In some embodiments, the method includes determining the predicted TH using a TH prediction model that receives a set of features in the sequence data as input and outputs the predicted TH, the set of features comprising at least one feature corresponding to one or more of: an allele frequency of single nucleotide variant (SNV) calls in the cfDNA sample, a mean allele frequency of cfDNA variants in the cfDNA sample, a ratio of minimum to maximum allele frequency of cfDNA variants in the cfDNA sample, and a reciprocal fraction of a number of cfDNA variants in the cfDNA sample.

In some embodiments, the TH prediction model comprises a linear regression model, and the method further comprises determining, with the TH prediction model, a coefficient of variation of the allele frequency of SNV calls based on the set of features; in accordance with a determination that the coefficient of variation is low, determining that the predicted TH is indicative of homogeneous tissue; and in accordance with a determination that the coefficient of variation is high, determining that the predicted TH is indicative of heterogeneous tissue.

In some embodiments, the TH prediction model comprises a statistical model trained on a training set comprising a plurality of training samples that are derived from cfDNA samples having matched tissue data from tumoral tissue samples, wherein training samples having high cfDNA-tissue concordance correspond to low coefficient of variation of cfDNA variant allele frequencies and are homogeneous, and training samples having low cfDNA-tissue concordance correspond to high coefficient of variation of cfDNA variant allele frequencies and are heterogeneous.

In some embodiments, the set of criteria includes a criterion that is met when the predicted TMB is high and a tumor fraction (TF) computed based on the sequence data is low. Further, in some embodiments, the method includes, subsequent to the determination that the predicted TMB is high, determining whether the TF is low, wherein the tumor fraction comprises a fraction of tumor-derived cfDNA over a total amount of cfDNA in the cfDNA sample; in accordance with a determination that the TF is low, determining that the subject is likely to respond to the treatment; and in accordance with a determination that the TF is not low, determining that the subject is not likely to respond to the treatment.

In some embodiments, the cfDNA sample is a blood-based sample.

In various embodiments, a device includes one or more processors; memory; and one or more programs, wherein the one or more programs are stored in the memory and configured to be executed by the one or more processors, the one or more programs including instructions for performing any of the methods described herein.

In accordance with some embodiments, an electronic device comprises means for performing any of the methods described herein.

In various embodiments, a non-transitory computer readable storage medium stores one or more programs, the one or more programs comprising instructions, which when executed by one or more processors of an electronic device, cause the device to perform any of the methods described above.

Executable instructions for performing these functions are, optionally, included in a transitory computer-readable storage medium or other computer program product configured for execution by one or more processors. In some embodiments, a transitory computer readable storage medium stores one or more programs comprising instructions, which when executed by one or more processors of an electronic device, cause the device to perform any of the methods described above.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the figures.

FIG. 1A is a flowchart of a method for preparing a nucleic acid sample for sequencing, according to various embodiments.

FIG. 1B is a graphical representation of the process for obtaining sequence reads, according to various embodiments.

FIG. 2 is a block diagram of a processing system for processing sequence reads, according to various embodiments.

FIG. 3 is a flowchart of a method for determining variants of sequence reads according to various embodiments.

FIG. 4 is a flow diagram illustrating an example method for predicting treatment response from cell-free DNA (“cfDNA”), according to various embodiments.

FIG. 5 is a schematic diagram of a processing system for predicting treatment response, according to various embodiments.

FIG. 6 is a plot showing a correlation between the TMB generated by whole-exome sequencing of tissue data and the TMB computed from a subset of regions of the exome data, according to various embodiments.

FIG. 7 is a diagram illustrating a feature matrix for training a model to predict TMB from blood-based data, according to various embodiments.

FIG. 8 is a plot showing the correlation between predicted TMB and ground truth TMB in a first investigation, according to various embodiments.

FIG. 9 is a plot showing consistent predictors of TMB in the first investigation, according to various embodiments.

FIG. 10 is a plot showing the correlation between predicted TMB and ground truth TMB in a second investigation, according to various embodiments.

FIG. 11 is a plot showing consistent predictors of TMB in the second investigation, according to various embodiments.

FIG. 12 is a plot showing cfDNA-tissue concordance plotted against the coefficient of variation (CV) of cfDNA allele frequencies (AFs), according to various embodiments.

FIG. 13 is a graph demonstrating performance of a model for distinguishing between homogeneous and heterogeneous samples with high TMB, according to various embodiments.

FIG. 14 is a graph demonstrating performance of the model of FIG. 13 on a set of all lung cancer samples, according to various embodiments.

FIG. 15 is a graph demonstrating performance of the model of FIG. 13 on all stage IV cancers, according to various embodiments.

FIG. 16 is a graph showing the overall survival of stage III and IV lung cancer patients that were treated with CIT versus other treatments, according to various embodiments.

FIG. 17 is a graph showing the use of PD-L1 negative expression as a biomarker for CIT benefit for stage III and IV lung cancer patients treated with CIT compared to other treatments, according to various embodiments.

FIG. 18 is a graph showing the use of PD-L1 positive expression as a biomarker for CIT benefit for stage III and IV lung cancer patients treated with CIT compared to other treatments, according to various embodiments.

FIG. 19 is a graph showing stage III and IV lung cancer patients treated with CIT versus other treatments for patients having a TMB=0, according to various embodiments.

FIG. 20 is a graph showing stage III and IV lung cancer patients treated with CIT versus other treatments for patients having a TMB between 0 and 10, according to various embodiments.

FIG. 21 is a graph showing stage III and IV lung cancer patients treated with CIT versus other treatments for patients having a TMB greater than or equal to 10, according to various embodiments.

FIG. 22 is a graph showing stage III and IV lung cancer patients treated with CIT versus other treatments, where the patients had a TF less than 1%, according to various embodiments.

FIG. 23 is a graph showing stage III and IV lung cancer patients treated with CIT versus other treatments, where the patients had a TF greater than or equal to 1%, according to various embodiments.

FIG. 24 is a graph showing stage III and IV lung cancer patients treated with CIT versus other treatments, where the patients had an ART estimated TF of less than 1%, according to various embodiments.

FIG. 25 shows stage III and IV lung cancer patients treated with CIT versus other treatments, where the patients had an ART estimated TF greater than or equal to 1%, according to various embodiments.

FIG. 26 depicts a block diagram of an example computer system, according to various embodiments.

### DETAILED DESCRIPTION

Reference will now be made in detail to several embodiments, examples of which are illustrated in the accompanying figures. It is noted that wherever practicable similar or like reference numbers may be used in the figures and may indicate similar or like functionality. Itis also noted that the contents of all published materials (patent applications, patents, papers, conference proceedings, and the like) referenced herein are incorporated herein by reference in their entirety.

### Definitions

Unless defined otherwise, all technical and scientific terms used herein have the meaning commonly understood by a person skilled in the art to which this description belongs. As used herein, the following terms have the meanings ascribed to them below.

The term “individual” refers to a human individual. The term “healthy individual” refers to an individual presumed to not have a cancer or disease.

The term “subject” refers to an individual whose DNA is being analyzed. A subject may be a test subject whose DNA is to be evaluated using whole genome sequencing or a targeted panel as described herein to evaluate whether the person has a disease state (e.g., cancer, type of cancer, or cancer tissue of origin). A subject may also be part of a control group known not to have cancer or another disease. A subject may also be part of a cancer or other disease group known to have cancer or another disease. Control and cancer/disease groups may be used to assist in designing or validating the targeted panel.

The term “reference sample” refers to a sample obtained from a subject with a known disease state.

The term “training sample” refers to a sample obtained from a known disease state that can be used to generate sequence reads. Training samples may be applied to probability models to generate features that can be utilized for disease state classification.

The term “test sample” refers to a sample that may have an unknown disease state.

The term “sequence read” refers to a nucleotide sequence read from a sample obtained from an individual. Sequence reads may be generated from nucleic acid fragments in the sample. A sequence read can be a collapsed sequence read generated from a plurality of sequence reads derived from a plurality of amplicons from a single original nucleic acid molecule. In some embodiments, the sequence read can be a deduplicated sequence read. Sequence reads can be obtained through various methods known in the art.

The term “read segment” or “read” refers to any nucleotide sequences including sequence reads obtained from an individual and/or nucleotide sequences derived from the initial sequence read from a sample obtained from an individual. For example, a read segment can refer to an aligned sequence read, a collapsed sequence read, or a stitched read. Furthermore, a read segment can refer to an individual nucleotide base, such as a single nucleotide variant.

The term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

The term “indel” refers to any insertion or deletion of one or more bases having a length and a position (which may also be referred to as an anchor position) in a sequence read. An insertion corresponds to a positive length, while a deletion corresponds to a negative length.

The term “mutation” refers to one or more SNVs or indels.

The term “candidate variant,” “called variant,” or “putative variant” refers to one or more detected nucleotide variants of a nucleotide sequence, for example, at a position in the genome that is determined to be mutated (i.e., a candidate SNV) or an insertion or deletion at one or more bases (i.e., a candidate indel). Generally, a nucleotide base is deemed a called variant based on the presence of an alternative allele on a sequence read, or collapsed read, where the nucleotide base at the position(s) differ from the nucleotide base in a reference genome. Additionally, candidate variants may be called as true positives or false positives.

The term “true positive” refers to a mutation that indicates real biology, for example, presence of a potential cancer, disease, or germline mutation in an individual. True positives are not caused by mutations naturally occurring in healthy individuals (e.g., recurrent mutations) or other sources of artifacts such as process errors during assay preparation of nucleic acid samples.

The term “false positive” refers to a mutation incorrectly determined to be a true positive. Generally, false positives may be more likely to occur when processing sequence reads associated with greater mean noise rates or greater uncertainty in noise rates.

The term “CpG site” refers to a region of a DNA molecule where a cytosine nucleotide is followed by a guanine nucleotide in the linear sequence of bases along its 5′ to 3′ direction. “CpG” is a shorthand for 5′-C-phosphate-G-3′ that is cytosine and guanine separated by only one phosphate group; phosphate links any two nucleotides together in DNA. Cytosines in CpG dinucleotides can be methylated to form 5-methylcytosine.

The term “methylation site” refers to a single site of a DNA molecule where a methyl group can be added. “CpG” sites are the most common methylation site, but methylation sites are not limited to CpG sites. For example, DNA methylation may occur in cytosines in CHG and CHH, where H is adenine, cytosine or thymine. Cytosine methylation in the form of 5-hydroxymethylcytosine may also assessed (see, e.g., WO 2010/037001 and WO 2011/127136, which are incorporated herein by reference), and features thereof, using the methods and procedures disclosed herein. The term “hypomethylated” or “hypermethylated” refers to a methylation status of a DNA molecule containing multiple CpG sites (e.g., more than 3, 4, 5, 6, 7, 8, 9, 10, etc.) where a high percentage of the CpG sites (e.g., more than 80%, 85%, 90%, or 95%, or any other percentage within the range of 50%-100%) are unmethylated or methylated, respectively.

The term “cell-free nucleic acids” or “cfNAs” refers to nucleic acid molecules that can be found outside cells, in bodily fluids such blood, sweat, urine, or saliva. Cell-free nucleic acids are used interchangeably as circulating nucleic acids.

The term “cell free nucleic acid,” “cell free DNA,” or “cfDNA” refers to deoxyribonucleic acid fragments that circulate in bodily fluids such blood, sweat, urine, or saliva and originate from one or more healthy cells and/or from one or more cancer cells.

The term “circulating tumor DNA” or “ctDNA” refers to deoxyribonucleic acid fragments that originate from tumor cells or other types of cancer cells, which may be released into an individual's bodily fluids such blood, sweat, urine, or saliva as result of biological processes such as apoptosis or necrosis of dying cells or actively released by viable tumor cells.

The term “circulating tumor RNA” or “ctRNA” refers to ribonucleic acid fragments that originate from tumor cells or other types of cancer cells, which may be released into an individual's bodily fluids such blood, sweat, urine, or saliva as result of biological processes such as apoptosis or necrosis of dying cells or actively released by viable tumor cells.

The term “genomic nucleic acid,” “genomic DNA,” or “gDNA” refers to nucleic acid including chromosomal DNA that originate from one or more healthy cells.

The term “alternative allele” or “ALT” refers to an allele having one or more mutations relative to a reference allele, e.g., corresponding to a known gene.

The term “sequencing depth” or “depth” refers to a total number of read segments from a sample obtained from an individual at a given position, region, or loci. In some embodiments, the depth refers to the average sequencing depth across the genome or across a targeted sequencing panel.

The term “alternate depth” or “AD” refers to a number of read segments in a sample that support an ALT, e.g., include mutations of the ALT.

The term “reference depth” refers to a number of read segments in a sample that include a reference allele at a candidate variant location.

The term “alternate frequency” or “AF” refers to the frequency of a given ALT. The AF may be determined by dividing the corresponding AD of a sample by the depth of the sample for the given ALT.

The term “variant” or “true variant” refers to a mutated nucleotide base at a position in the genome. Such a variant can lead to the development and/or progression of cancer in an individual.

The term “disease state” refers to presence or non-presence of a disease, a type of disease, and/or a disease tissue of origin. For example, in one embodiment, the present disclosure provides methods, systems, and non-transitory computer readable medium for detecting cancer (i.e., presence or absence of cancer), a type of cancer, or a cancer tissue of origin.

The term “tissue of origin” or “TOO” refers to the organ, organ group, body region or cell type from which a disease state may arise or originate. For example, the identification of a tissue of origin or cancer cell type typically allows to identify appropriate next steps to further diagnose, stage, and decide on treatment.

The term “tumor mutational burden (TMB)” refers to the total number of mutations (changes) found in the DNA of cancer cells. In practice, TMB can be defined in several ways, including a total number of nonsynonymous point mutations for a sample (e.g., cancer tissue sample) or a total number of variants per individual that are called as candidate variants in the individual's cfDNA sample. In some cases, TMB is defined as a total number of nonsynonymous point mutations divided by a total number of mutations in the exome, and/or per megabase (e.g., divided by a total number of megabases), and/or including or excluding indels. Tumors with cells that have a high number of mutations (a high TMB) can be more likely to respond to certain types of immunotherapy. In this way, TMB can be used as a type of predictive biomarker for response to certain immuno-oncology (I-O) therapy.

The term “tumor heterogeneity (TH)” refers to differences between cancer cells within a tumor or within multiple tumors in a single patient. Intra-tumor heterogeneity refers to the presence of more than one clone of cancer cells within a given tumor mass, while inter-tumor heterogeneity refers to the presence of different genetic alterations in different metastatic tumors from a single patient.

The term “tumor fraction (TF)” refers to the fraction of cfDNA derived from tumor cells. For example, TF is the amount of ctDNA to the total cfDNA in a patient sample.

### Overview

Immunotherapy is a major breakthrough in cancer treatment. However, only a subset of patients respond to certain types of immunotherapies. Some techniques for predicting whether a patient will respond to immunotherapy include acquiring tumor tissue samples via tissue biopsies from the patient. Such tissue samples can be analyzed by immunohistochemistry and/or sequencing analysis (e.g., whole-exome sequencing of nucleic acids derived from the tissue sample) to assess the tumor mutational burden (TMB) of the sample. TMB refers to the total number of mutations (changes) found in the DNA of cancer cells, and can provide insight to the level of benefit the patient would receive from an immunotherapy treatment. For instance, tumors having a high number of mutations (a high TMB) appear to be more likely to respond to certain types of immunotherapy, while tumors having low TMB are less likely to respond to immunotherapy. While TMB based on tissue samples can be used for assessing whether a patient will benefit from an immunotherapy treatment, unfortunately, tissue biopsies are invasive and may not be available to all patients.

The present disclosure provides improved techniques for predicting or monitoring treatment response to immunotherapy in the absence of tissue samples. Specifically, systems and methods disclosed herein provide a liquid biopsy-based assessment of one or more biomarkers indicative of treatment response. For instance, some methods disclosed herein are directed to predicting a TMB of a tumoral tissue based on sequencing data of a cell-free DNA (“cfDNA”) sample (e.g., a blood sample) obtained from a patient. As described herein, the predicted TMB from the cfDNA sample is used to assess whether the patient is likely to respond to immunotherapy, such as checkpoint inhibition treatments. In some methods disclosed herein, predicting or otherwise assessing the patient's treatment response includes determining a tumoral heterogeneity (“TH”) of the tissue based on the cfDNA data. Further, some methods described herein include assessing tumor fraction (“TF”) from the cfDNA data to assess the treatment response.

By determining biomarkers such as TMB, TH, TF, and/or any combination thereof from cfDNA samples gathered using noninvasive and widely available techniques, such as a blood draw, the present disclosure provides significant improvements for predicting and monitoring a patient's treatment response to immunotherapy. For instance, the blood-based assessments described herein can provide faster, more accurate and/or more informative results than traditional techniques, and therefore can lower costs and enhance treatment efficacy by identifying appropriate treatment plans for patients. Such techniques can be used to determine whether a patient is a candidate for a certain immunotherapy before it is administered. Further, the systems and methods described herein can be utilized to monitor a patient's responsiveness to an ongoing treatment and assess whether the treatment should be altered or adjusted during the course of its administration. Because blood samples are relatively non-invasive and easy to obtain compared to tissue biopsies, in some cases, several blood samples can be drawn from a patient at different time points while a treatment is being administered, such that cfDNA data gathered from the samples can be evaluated throughout the course of administration to determine whether the patient is responding to the treatment and whether to alter the treatment. Overall, such improvements can decrease the mortality rate of cancer patients by saving critical time in identifying effective treatment plans for each patient and monitoring the effectiveness of treatment plans during their administration. Additional advantages are contemplated and described further below.

It is noted that while the systems and methods disclosed herein are envisioned to be used as an alternative to existing methods, such as invasive methods requiring tissue biopsies, in some examples, the systems and methods herein can also be used in conjunction with such existing methods or as a companion diagnostic tool along with such methods. Additionally, while the present disclosure describes measuring (or otherwise determining, estimating, and/or predicting measurements for) TMB, TH, and TF from cfDNA, it is contemplated that other predictive biomarkers can be determined or otherwise estimated from cell-free nucleic acid (“cfNA”) data, such as other biomarkers indicative of treatment response for a variety of immunotherapies, including immuno oncology (“IO”) treatment. While the techniques described herein employ data from cfDNA, data from other cfNAs such as cell-free RNA can be gathered and implemented, additionally or alternatively to cfDNA data.

Example Assay Protocol

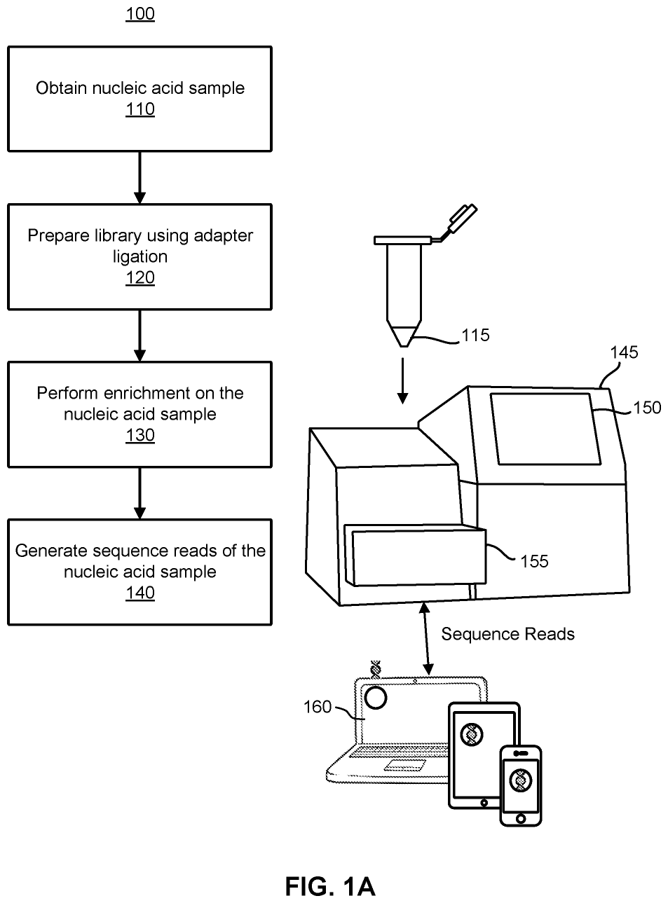

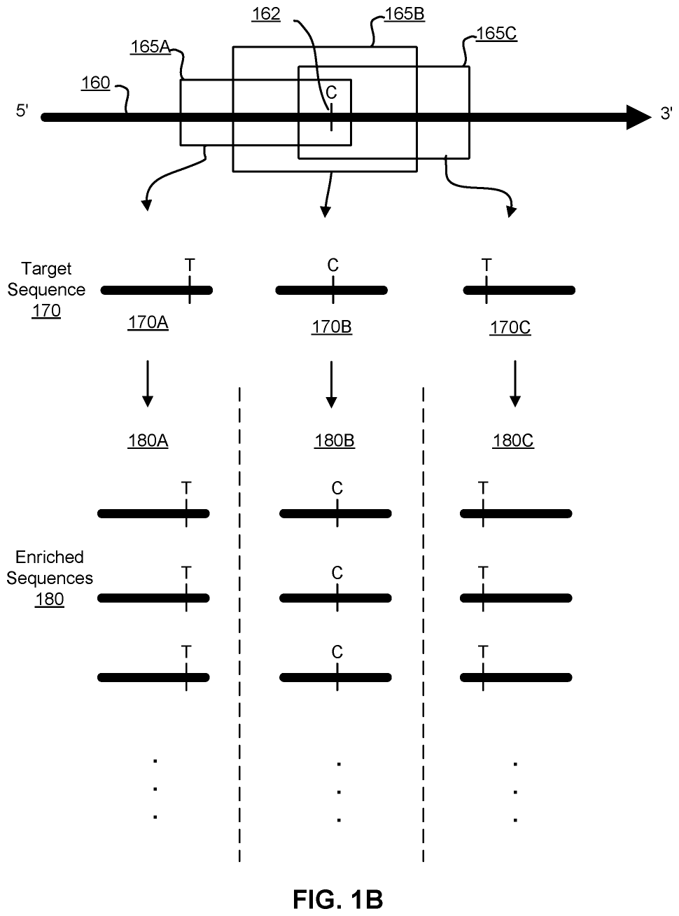

FIG. 1A is flowchart of a method 100 for preparing a nucleic acid sample for sequencing according to some embodiments. The method 100 includes, but is not limited to, the following steps. For example, any step of the method 100 can comprise a quantitation sub-step for quality control or other laboratory assay procedures known to one skilled in the art.

In step 110, a test sample comprising a plurality of nucleic acid molecules (DNA or RNA) is obtained from a subject, and the nucleic acids are extracted and/or purified from the test sample. In the present disclosure, DNA and RNA can be used interchangeably unless otherwise indicated. That is, the following embodiments for using error source information in variant calling and quality control can be applicable to both DNA and RNA types of nucleic acid sequences. However, the examples described herein may focus on DNA for purposes of clarity and explanation. The nucleic acids in the extracted sample can comprise the whole human genome, or any subset of the human genome, including the whole exome. Alternatively, the sample can be any subset of the human transcriptome, including the whole transcriptome. The test sample can be obtained from a subject known to have or suspected of having cancer. In some embodiments, the test sample can include blood, plasma, serum, urine, fecal, saliva, other types of bodily fluids, or any combination thereof. Alternatively, the test sample can comprise a sample selected from the group consisting of whole blood, a blood fraction, a tissue biopsy, pleural fluid, pericardial fluid, cerebral spinal fluid, and peritoneal fluid. In some embodiments, methods for drawing a blood sample (e.g., syringe or finger prick) are less invasive than procedures for obtaining a tissue biopsy, which may require surgery. The extracted sample can comprise cfDNA and/or ctDNA. For healthy individuals, the human body may naturally clear out cfDNA and other cellular debris. In general, any known method in the art can be used to extract and purify cell-free nucleic acids from the test sample. For example, cell-free nucleic acids can be extracted and purified using one or more known commercially available protocols or kits, such as the QIAamp circulating nucleic acid kit (QIAGEN®). If a subject has a cancer or disease, ctDNA in an extracted sample may be present at a detectable level for diagnosis.

In step 120, a sequencing library is prepared. During library preparation, sequencing adapters comprising unique molecular identifiers (UMI) are added to the nucleic acid molecules (e.g., DNA molecules), for example, through adapter ligation (using T4 or T7 DNA ligase) or other known means in the art. The UMIs are short nucleic acid sequences (e.g., 4-10 base pairs) that are added to ends of DNA fragments and serve as unique tags that can be used to identify nucleic acids (or sequence reads) originating from a specific DNA fragment. Following adapter addition, the adapter-nucleic acid constructs are amplified, for example, using polymerase chain reaction (PCR). During PCR amplification, the UMIs are replicated along with the attached DNA fragment, which provides a way to identify sequence reads that came from the same original fragment in downstream analysis. Optionally, as is well known in the art, the sequencing adapters may further comprise a universal primer, a sample-specific barcode (for multiplexing) and/or one or more sequencing oligonucleotides for use in subsequent cluster generation and/or sequencing (e.g., known P5 and P7 sequences for used in sequencing by synthesis (SBS) (ILLUMINA®, San Diego, Calif.)).

In step 130, targeted DNA sequences are enriched from the library. In accordance with some embodiments, during targeted enrichment, hybridization probes (also referred to herein as “probes”) are used to target, and pull down, nucleic acid fragments known to be, or that may be, informative for the presence or absence of cancer (or disease), cancer status, or a cancer classification (e.g., cancer type or tissue of origin). For a given workflow, the probes can be designed to anneal (or hybridize) to a target (complementary) strand of DNA or RNA. The target strand can be the “positive” strand (e.g., the strand transcribed into mRNA, and subsequently translated into a protein) or the complementary “negative” strand. The probes can range in length from 10s, 100s, or 1000s of base pairs. In one embodiment, the probes are designed based on a gene panel to analyze particular mutations or target regions of the genome (e.g., of the human or another organism) that are suspected to correspond to certain cancers or other types of diseases. Moreover, the probes can cover overlapping portions of a target region. As one of skill in the art would readily appreciate, any known means in the art can be used for targeted enrichment. For example, in some embodiments, the probes may be biotinylated and streptavidin coated magnetic beads used to enrich for probe captured target nucleic acids. See, e.g., Duncavage et al., J Mol Diagn. 13(3): 325-333 (2011); and Newman et al., Nat Med. 20(5): 548-554 (2014). By using a targeted gene panel rather than sequencing the whole genome (“whole genome sequencing”), all expressed genes of a genome (“whole exome sequencing” or “whole transcriptome sequencing”), the method 100 can be used to increase sequencing depth of the target regions, where depth refers to the count of the number of times a given target sequence within the sample has been sequenced. Increasing sequencing depth allows for detection of rare sequence variants in a sample and/or increases the throughput of the sequencing process. After a hybridization step, the hybridized nucleic acid fragments are captured and can also be amplified using PCR.

Turning now to FIG. 1B, FIG. 1B is a graphical representation of the process for obtaining sequence reads according to some embodiments. FIG. 1B depicts an example of a nucleic acid segment 160 from the sample. Here, the nucleic acid segment 160 can be a single-stranded nucleic acid segment, such as a single stranded DNA or single stranded RNA segment. In some embodiments, the nucleic acid segment 160 is a double-stranded cfDNA segment. The illustrated example depicts three regions 165A, 165B, and 165C of the nucleic acid segment 160 that can be targeted by different probes. Specifically, each of the three regions 165A, 165B, and 165C includes an overlapping position on the nucleic acid segment 160. An example overlapping position is depicted in FIG. 1B as the cytosine (“C”) nucleotide base 162. The cytosine nucleotide base 162 is located near a first edge of region 165A, at the center of region 165B, and near a second edge of region 165C.

In some embodiments, one or more (or all) of the probes are designed based on a gene panel to analyze particular mutations or target regions of the genome (e.g., of the human or another organism) that are suspected to correspond to certain cancers or other types of diseases. By using a targeted gene panel rather than sequencing all expressed genes of a genome, also known as “whole exome sequencing,” the method 100 can be used to increase sequencing depth of the target regions, where depth refers to the count of the number of times a given target sequence within the sample has been sequenced. Increasing sequencing depth reduces required input amounts of the nucleic acid sample.

Hybridization of the nucleic acid sample 160 using one or more probes results in an understanding of a target sequence 170. As shown in FIG. 1B, the target sequence 170 is the nucleotide base sequence of the region 165 that is targeted by a hybridization probe. The target sequence 170 can also be referred to as a hybridized nucleic acid fragment. For example, target sequence 170A corresponds to region 165A targeted by a first hybridization probe, target sequence 170B corresponds to region 165B targeted by a second hybridization probe, and target sequence 170C corresponds to region 165C targeted by a third hybridization probe. Given that the cytosine nucleotide base 162 is located at different locations within each region 165A-C targeted by a hybridization probe, each target sequence 170 includes a nucleotide base that corresponds to the cytosine nucleotide base 162 at a particular location on the target sequence 170.

In the example of FIG. 1B, the target sequence 170A and target sequence 170C each have a nucleotide base (shown as thymine “T”) that is located near the edge of the target sequences 170A and 170C. Here, the thymine nucleotide base (e.g., as opposed to a cytosine base) may be a result of a random cytosine deamination process that causes a cytosine base to be subsequently recognized as a thymine nucleotide base during the sequencing process. Thus, the C>T SNV for target sequences 170A and 170C may be considered an edge variant because the mutation is located at an edge of target sequences 170A and 170C. A cytosine deamination process can lead to a downstream sequencing artifact that prevents the accurate capture of the actual nucleotide base pair in the nucleic acid segment 160. Additionally, target sequence 170B has a cytosine base that is located at the center of the target sequence 170B. Here, a cytosine base that is located at the center may be less susceptible to cytosine deamination.

After a hybridization step, the hybridized nucleic acid fragments are captured and may also be amplified using PCR. For example, the target sequences 170 can be enriched to obtain enriched sequences 180 that can be subsequently sequenced. In some embodiments, each enriched sequence 180 is replicated from a target sequence 170. Enriched sequences 180A and 180C that are amplified from target sequences 170A and 170C, respectively, also include the thymine nucleotide base located near the edge of each sequence read 180A or 180C. As used hereafter, the mutated nucleotide base (e.g., thymine nucleotide base) in the enriched sequence 180 that is mutated in relation to the reference allele (e.g., cytosine nucleotide base 162) is considered as the alternative allele. Additionally, each enriched sequence 180B amplified from target sequence 170B includes the cytosine nucleotide base located near or at the center of each enriched sequence 180B.

Turning back to FIG. 1A, in step 140, sequence reads are generated from the enriched nucleic acid molecules (e.g., DNA molecules). Sequencing data or sequence reads can be acquired from the enriched nucleic acid molecules by known means in the art. For example, the method 100 can include next generation sequencing (NGS) techniques including synthesis technology (ILLUMINA®), pyrosequencing (454 LIFE SCIENCES), ion semiconductor technology (Ion Torrent sequencing), single-molecule real-time sequencing (PACIFIC BIOSCIENCES®), sequencing by ligation (SOLiD sequencing), nanopore sequencing (OXFORD NANOPORE TECHNOLOGIES), or paired-end sequencing. In some embodiments, massively parallel sequencing is performed using sequencing-by-synthesis with reversible dye terminators.

In various embodiments, the enriched nucleic acid sample 115 is provided to the sequencer 145 for sequencing. As shown in FIG. 1A, the sequencer 145 can include a graphical user interface 150 that enables user interactions with particular tasks (e.g., initiate sequencing or terminate sequencing) as well as one more loading trays 155 for providing the enriched fragment samples and/or necessary buffers for performing the sequencing assays. Therefore, once a user has provided the necessary reagents and enriched fragment samples to the loading trays 155 of the sequencer 145, the user can initiate sequencing by interacting with the graphical user interface 150 of the sequencer 145. In step 140, the sequencer 145 performs the sequencing and outputs the sequence reads of the enriched fragments from the nucleic acid sample 115.

In some embodiments, the sequencer 145 is communicatively coupled with one or more computing devices 160. Each computing device 160 can process the sequence reads for various applications such as variant calling or quality control. The sequencer 145 can provide the sequence reads in a BAM file format to a computing device 160. Each computing device 160 can be one of a personal computer (PC), a desktop computer, a laptop computer, a notebook, a tablet PC, or a mobile device. A computing device 160 can be communicatively coupled to the sequencer 145 through a wireless, wired, or a combination of wireless and wired communication technologies. Generally, the computing device 160 is configured with a processor and memory storing computer instructions that, when executed by the processor, cause the processor to process the sequence reads or to perform one or more steps of any of the methods or processes disclosed herein.

In some embodiments, the sequence reads can be aligned to a reference genome using known methods in the art to determine alignment position information. For example, in some embodiments, sequence reads are aligned to human reference genome hg19. The sequence of the human reference genome, hg19, is available from Genome Reference Consortium with a reference number, GRCh37/hg19, and also available from Genome Browser provided by Santa Cruz Genomics Institute. The alignment position information can indicate a beginning position and an end position of a region in the reference genome that corresponds to a beginning nucleotide base and end nucleotide base of a given sequence read. Alignment position information can also include sequence read length, which can be determined from the beginning position and end position. A region in the reference genome can be associated with a gene or a segment of a gene.

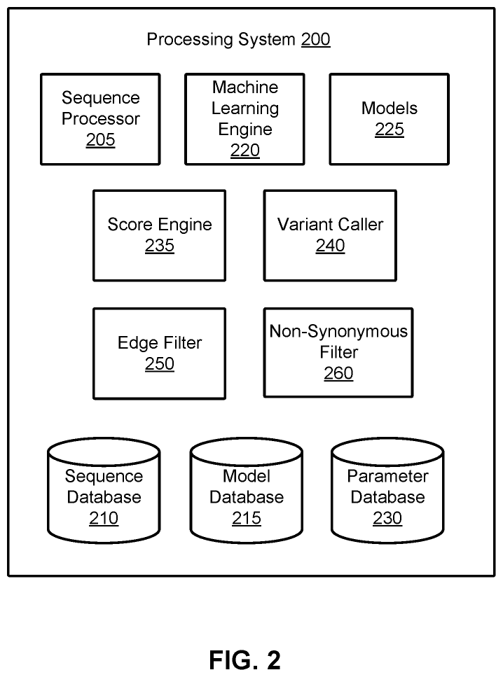

In various embodiments, for example when a paired-end sequencing process is used, a sequence read is comprised of a read pair denoted as R₁ and R₂. For example, the first read R₁ can be sequenced from a first end of a double-stranded DNA (dsDNA) molecule whereas the second read R₂ can be sequenced from the second end of the double-stranded DNA (dsDNA). Therefore, nucleotide base pairs of the first read R₁ and second read R₂ can be aligned consistently (e.g., in opposite orientations) with nucleotide bases of the reference genome. Alignment position information derived from the read pair R₁ and R₂ can include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format can be generated and output for further analysis such as variant calling, as described below with respect to FIG. 2.

Example Processing System for Processing Sequence Reads

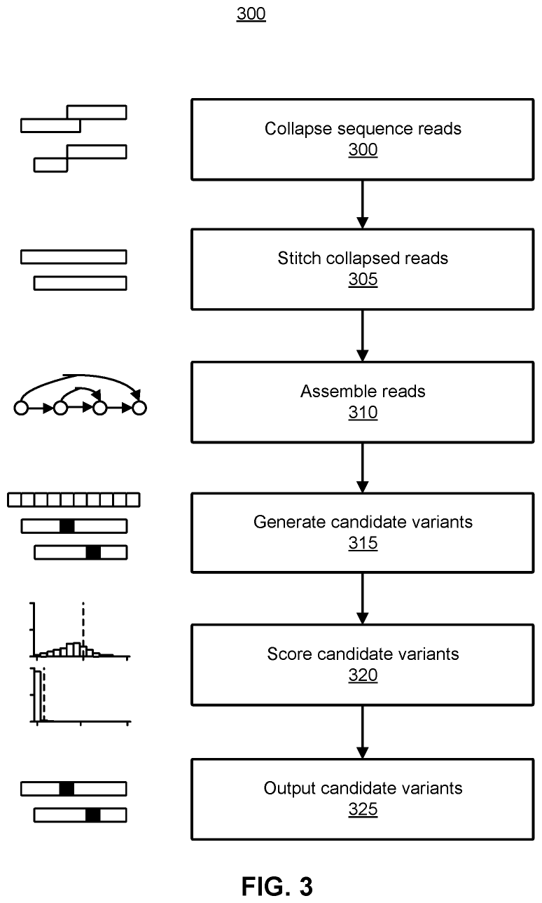

Turning now to FIGS. 2-3, FIG. 2 is a block diagram of a processing system 200 for processing sequence reads according to some embodiments. The processing system 200 includes a sequence processor 205, sequence database 210, model database 215, machine learning engine 220, models 225 (for example, including a “Bayesian hierarchical model” or a “predictive cancer model”), parameter database 230, score engine 235, variant caller 240, edge filter 250, and non-synonymous filter 260. FIG. 3 is flowchart of a method 300 for determining variants of sequence reads according to some embodiments. In some embodiments, the processing system 200 performs the method 300 to perform variant calling (e.g., for SNVs and/or indels) based on input sequencing data. Further, the processing system 200 can obtain the input sequencing data from an output file associated with nucleic acid sample prepared using the method 100 described above. The method 300 includes, but is not limited to, the following steps, which are described with respect to the components of the processing system 200. In other embodiments, one or more steps of the method 300 can be replaced by a step of a different process for generating variant calls, e.g., using Variant Call Format (VCF), such as HaplotypeCaller, VarScan, Strelka, or SomaticSniper.

At step 300, optionally, the sequence processor 205 collapses aligned sequence reads of the input sequencing data. In some embodiments, collapsing sequence reads includes using UMIs, and optionally alignment position information from sequencing data of an output file (e.g., from the method 100 shown in FIG. 1A) to identify and collapse multiple sequence reads (i.e., derived from the same original nucleic acid molecule) into a consensus sequence. In accordance with this step, a consensus sequence is determined from multiple sequence reads derived from the same original nucleic acid molecule that represents the most likely nucleic acid sequence, or portion thereof, of the original molecule. Since the UMI sequences are replicated through PCR amplification of the sequencing library, the sequence processor 205 can determine that certain sequence reads originated from the same molecule in a nucleic acid sample. In some embodiments, sequence reads that have the same or similar alignment position information (e.g., beginning and end positions within a threshold offset) and include a common UMI are collapsed, and the sequence processor 205 generates a collapsed read (also referred to herein as a consensus read) to represent the nucleic acid fragment. In some embodiments, the sequence processor 205 designates a consensus read as “duplex” if the corresponding pair of sequence reads (i.e., R₁ and R₂), or collapsed sequence reads, have a common UMI, which indicates that both positive and negative strands of the originating nucleic acid molecule have been captured; otherwise, the collapsed read is designated “non-duplex.” In some embodiments, the sequence processor 205 can perform other types of error correction on sequence reads as an alternative to, or in addition to, collapsing sequence reads.

At step 305, optionally, the sequence processor 205 can stitch sequence reads, or collapsed sequence reads, based on the corresponding alignment position information merging together two sequence reads into a single read segment. In some embodiments, the sequence processor 205 compares alignment position information between a first sequence read and a second sequence read (or collapsed sequence reads) to determine whether nucleotide base pairs of the first and second reads partially overlap in the reference genome. In one use case, responsive to determining that an overlap (e.g., of a given number of nucleotide bases) between the first and second reads is greater than a threshold length (e.g., threshold number of nucleotide bases), the sequence processor 205 designates the first and second reads as “stitched”; otherwise, the collapsed reads are designated “unstitched.” In some embodiments, a first and second read are stitched if the overlap is greater than the threshold length and if the overlap is not a sliding overlap. For example, a sliding overlap can include a homopolymer run (e.g., a single repeating nucleotide base), a dinucleotide run (e.g., two-nucleotide repeating base sequence), or a trinucleotide run (e.g., three-nucleotide repeating base sequence), where the homopolymer run, dinucleotide run, or trinucleotide run has at least a threshold length of base pairs.

At step 310, the sequence processor 205 can optionally assemble two or more reads, or read segments, into a merged sequence read (or a path covering the targeted region). In some embodiments, the sequence processor 205 assembles reads to generate a directed graph, for example, a de Bruijn graph, for a target region (e.g., a gene). Unidirectional edges of the directed graph represent sequences of k nucleotide bases (also referred to herein as “k-mers”) in the target region, and the edges are connected by vertices (or nodes). The sequence processor 205 aligns collapsed reads to a directed graph such that any of the collapsed reads may be represented in order by a subset of the edges and corresponding vertices.

In some embodiments, the sequence processor 205 determines sets of parameters describing directed graphs and processes directed graphs. Additionally, the set of parameters may include a count of successfully aligned k-mers from collapsed reads to a k-mer represented by a node or edge in the directed graph. The sequence processor 205 stores, e.g., in the sequence database 210, directed graphs and corresponding sets of parameters, which can be retrieved to update graphs or generate new graphs. For instance, the sequence processor 205 can generate a compressed version of a directed graph (e.g., or modify an existing graph) based on the set of parameters. In one use case, in order to filter out data of a directed graph having lower levels of importance, the sequence processor 205 removes (e.g., “trims” or “prunes”) nodes or edges having a count less than a threshold value, and maintains nodes or edges having counts greater than or equal to the threshold value.

At step 315, the variant caller 240 generates candidate variants from the sequence reads, collapsed sequence reads, or merged sequence reads assembled by the sequence processor 205. In some embodiments, the variant caller 240 generates the candidate variants by comparing sequence reads, collapsed sequence reads, or merged sequence reads (which may have been compressed by pruning edges or nodes in step 310) to a reference sequence of a target region of a reference genome (e.g., human reference genome hg19). The variant caller 240 can align edges of the sequence reads collapsed sequence reads, or merged sequence reads to the reference sequence, and records the genomic positions of mismatched edges and mismatched nucleotide bases adjacent to the edges as the locations of candidate variants. In some embodiments, the genomic positions of mismatched nucleotide bases to the left and right edges are recorded as the locations of called variants. Additionally, the variant caller 240 can generate candidate variants based on the sequencing depth of a target region. In particular, the variant caller 240 can be more confident in identifying variants in target regions that have greater sequencing depth, for example, because a greater number of sequence reads help to resolve (e.g., using redundancies) mismatches or other base pair variations between sequences.

In some embodiments, the variant caller 240 generates candidate variants using the model 225 to determine expected noise rates for sequence reads from a subject (e.g., from a healthy subject). The model 225 can be a Bayesian hierarchical model, though in some embodiments, the processing system 200 uses one or more different types of models. Moreover, a Bayesian hierarchical model can be one of many possible model architectures that may be used to generate candidate variants and which are related to each other in that they all model position-specific noise information in order to improve the sensitivity or specificity of variant calling. More specifically, the machine learning engine 220 trains the model 225 using samples from healthy individuals to model the expected noise rates per position of sequence reads.

Further, multiple different models can be stored in the model database 215 or retrieved for application post-training. For example, a first model is trained to model SNV noise rates and a second model is trained to model indel noise rates. Further, the score engine 235 can use parameters of the model 225 to determine a likelihood of one or more true positives in a sequence read. The score engine 235 can determine a quality score (e.g., on a logarithmic scale) based on the likelihood. For example, the quality score is a Phred quality score Q=−10·log₁₀ P, where P is the likelihood of an incorrect candidate variant call (e.g., a false positive).

At step 320, the score engine 235 scores the candidate variants based on the model 225 or corresponding likelihoods of true positives or quality scores. Training and application of the model 225 is described in more detail in U.S. patent application Ser. No. 16/201,912, entitled “Models for Targeted Sequencing,” and filed on Nov. 27, 2018, the content of which is incorporated herein by reference in its entirety. In some embodiments, the processing system 200 can filter the candidate variants using one or more criteria. For example, processing system 200 filter candidate variants having at least (or less than) a threshold score.

At step 325, the processing system 200 outputs the candidate variants. In some embodiments, the processing system 200 outputs some or all of the determined candidate variants along with the corresponding scores. Downstream systems, e.g., external to the processing system 200 or other components of the processing system 200, can use the candidate variants and scores for various applications including, but not limited to, predicting presence of cancer, disease, or germline mutations.

FIGS. 1-3 exemplify possible embodiments for generating sequencing read data and identifying candidate variants or rare mutation calls. However, as one of skill in the art would readily appreciate, other known means in the art for obtaining sequencing data, such as sequence reads or consensus sequence reads, and identifying candidate variants or rare mutation calls therefrom, can be used in the practice of embodiments of the present invention (see, e.g., U.S. Patent Publication No. 2012/0065081, U.S. Patent Publication No. 2014/0227705, U.S. Patent Publication No. 2015/0044687 and U.S. Patent Publication No. 2017/0058332).

Predicting Tumor Mutational Burden (“TMB”) from cfDNA to Determine Treatment Response

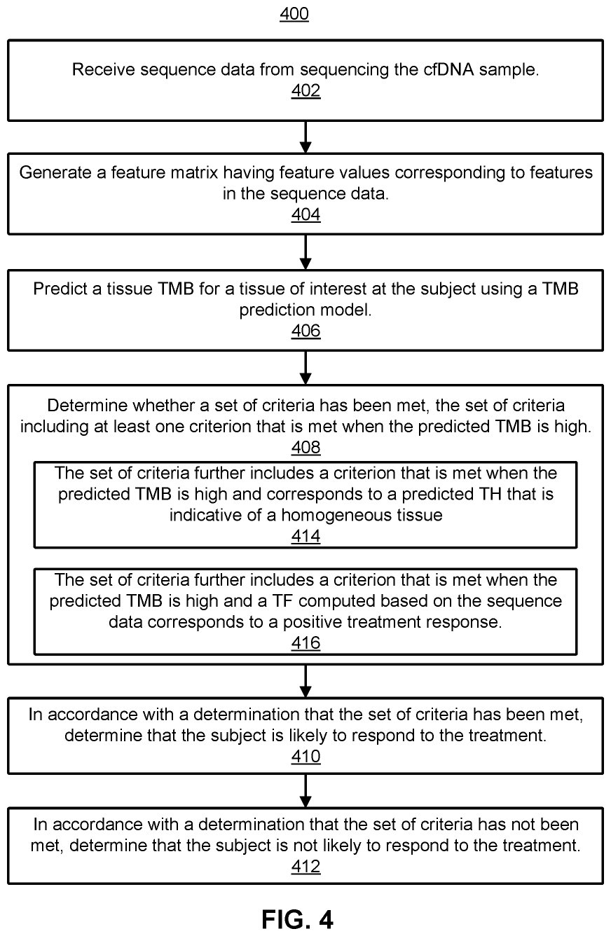

FIG. 4 illustrates an example method 400 for predicting treatment response from cfDNA data. The method 400 estimates cancer tissue TMB from a cfDNA sample (e.g., a blood sample) and utilizes the TMB as a non-invasive biomarker for IO treatment. For instance, the TMB can be used to determine whether a cancer patient, and more specifically whether a tumor at the cancer patient, is likely to respond to immunotherapy, such as IO drugs (e.g., anti-PD1 or anti-PDL1 inhibitors). As discussed below, the TMB can be predicted based on a combination of single nucleotide variants (“SNVs”), somatic copy number aberrations (“SCNAs”), and/or DNA methylation signals. Other features can be utilized, additionally and/or alternatively, for predicting cancer tissue TMB. Method 400 includes, but is not limited to, the following steps.

Method 400 includes, at block 402, receiving sequence data gathered from sequencing a cfDNA sample (e.g., blood sample) obtained from a subject. The subject can be a patient suspected of having, at risk of having, or known to have a disease state, such as cancer.

It is noted that while method 100 is described using a cfDNA sample, other test samples can be utilized, such as other samples containing a plurality of nucleic acids (e.g., a plurality of cfNAs including cfDNA or cell-free RNA (“cfRNA”)) originating from healthy cells and/or unhealthy cells (e.g., cancer cells). Examples of other test samples containing cfNAs can include, merely by way of example, a biological fluid sample selected from the group consisting of blood, plasma, serum, urine, saliva, fecal samples, and any combination thereof. In some examples, the test sample or biological test sample comprises a test sample selected from the group consisting of one or more blood cells, whole blood, a blood fraction, plasma, serum, pleural fluid, pericardial fluid, cerebrospinal fluid, peritoneal fluid, urea, sweat, saliva, tears, fecal material, and any combination thereof. In some examples, the sample is a plasma sample from a cancer patient, or a patient suspected of having cancer.

The sequence data or sequence reads from the cfDNA sample can be generated by sequencing the cfDNA sample using any means known in the art. Example sequencing techniques are described above in relation to FIGS. 1-3. In some examples, the sequence data is obtained by whole-genome sequencing (“WGS”), whole-genome bisulfite sequencing (“WGBS”), and/or whole-exome sequencing (“WES”). In some examples, the test sample includes a plurality of cfRNA, and sequencing is RNA sequencing (RNA-seq), transcriptome sequencing or whole-transcriptome shotgun sequencing (WTSS). For RNA sequencing, it is common to convert isolated RNA molecules to complementary DNA (cDNA) molecules using reverse transcriptase, prior to library preparation and sequencing. In some examples, the sequencing library is sequenced to a depth of at least 10×, at least 20×, at least 30×, at least 50×, or at least 100×. In other examples, the sequencing library is sequenced to a depth of at least 500×, at least 1,000×, at least 2,000×, at least 3,000×, or at least 10,000×.

Additionally, while method 400 is directed to prediction of treatment response for cancer immunotherapy, it is noted that other types of therapies can be evaluated for patients suspected of having, at risk of having, or known to have other types of disease states. Such disease states can include, but are not limited to, cardiovascular disease, neurodegenerative disease, or other disease.

Referring again to FIG. 4, at block 404, method 400 includes generating a feature matrix comprising feature values corresponding to synonymous and nonsynonymous mutations in the sequence data. The feature values can represent features including, but not limited to, one or more of: a number of nonsynonymous somatic mutations for each region of a plurality of regions included in an assay used to sequence the cfDNA sample, a total number of somatic mutations in the sample, a total number of nonsynonymous somatic mutations in the sample, an allele frequency (“AF”) of cfDNA variants in the sample, a sum of the AFs, and/or any combinations thereof.

Feature values in the feature matrix can be derived from the sequence data. In some examples, the sequence data is generated by a sequencing assay or panel, such as a targeted sequencing assay, having a plurality of regions or genomic regions. Each region on the panel can correspond to an individual gene. In such examples, the feature matrix can represent features corresponding to the plurality of genes in the assay. For instance, the feature matrix can include a number of nonsynonymous somatic mutations for each gene of the sequencing panel. In some examples, the sequence data is filtered or cleaned prior to generating the feature matrix, such that the feature matrix represents values from cleaned sequence data. The plurality of genes represented in the feature matrix can include a subset of the full set of genes in the sequencing assay. For example, after the data is cleaned, a subset of the genes in the sequence data can be analyzed for nonsynonymous mutations.

In some embodiments, the feature matrix comprises a plurality of positions that include at least one position for each gene to represent a value or number of nonsynonymous somatic mutations at that gene. In some examples, the plurality of positions further include a position for a total number of somatic mutations in the sample, and/or a position for a total number of nonsynonymous somatic mutations in the sample. Still, in some examples, the feature matrix represents features from sequence data from a plurality of test samples, such as a plurality of cfDNA samples. Variations in the feature matrix can be contemplated without departing from the spirit of the invention.

The feature values can be derived by analyzing the sequence data using any known means in the art, such as means for detecting and quantifying mutations (e.g., somatic mutations or variants at a locus or at a plurality of loci). For example, a variant calling pipeline can be used to detect and quantify somatic mutations or variants. See, e.g., U.S. patent application Ser. No. 16/201,912, entitled “Models for Targeted Sequencing,” and filed on Nov. 27, 2018, and International Patent Application No. PCT/US20/48448, entitled “Systems and Methods for Determining Consensus Base Calls in Nucleic Acid Sequencing,” and filed on Aug. 28, 2020, the contents of which are incorporated herein by reference in their entirety. See also, e.g., Brockman et al., 2008 Genome Res 187, 763-770; Ledergerber et al., 2011 Briefings in Bioinformatics 12(5), 489-497; Snyder et al., 2016 Cell 164, 57-68. A noise model can be applied to account for noise in the estimated feature values or features. See, e.g., U.S. patent application Ser. No. 16/153,593, entitled “Site-Specific Noise Model For Targeted Sequencing,” and filed on Oct. 5, 2018, the content of which is incorporated herein by reference in its entirety. In some examples, one or more white blood cell (“WBC”) derived somatic mutations can be detected, identified, or otherwise accounted for. See, e.g., U.S. patent application Ser. No. 16/417,336, entitled “Inferring Selection in White Blood Cell Matched Cell-Free DNA Variants and/or RNA Variants,” and filed on May 20, 2019, the content of which is incorporated herein by reference in its entirety.

In some examples, sequence reads covering one or more loci or genes known to be associated with a disease state can be analyzed to detect somatic mutations or variants at the loci or genes. Such loci or genes can be known to be, or suspected of being, associated with cancer, such as a particular type of cancer or tumoral tissue. In some examples, sequence reads can be analyzed for identification of a known somatic mutation in a subject (e.g., a known somatic mutation associated with a disease or disease state) to assess or infer how a subject will respond to a therapeutic treatment targeting that somatic mutation. In still cases, sequence reads can be analyzed to identify previously unknown, or previously undetected somatic mutations (or variants) as potential targets for development of a therapeutic agent to treat a particular disease or disease state.

In some examples, somatic mutations can comprise single-nucleotide variants, small insertions and/or deletions (“indels”). For instance, the one or more somatic mutations can comprise one or more nonsynonymous mutations, one or more missense mutations, one or more nonsense mutations, one or more truncating mutations, and/or one or more essential splice site mutations.

Further, in some examples, the feature values can be based on methylation signals in the cfDNA, and more particularly on anomalously methylated fragments identified in the cfDNA. For instance, anomalous fragments can be identified as fragments with over a threshold number of CpG sites and either with over a threshold percentage of the CpG sites methylated or with over a threshold percentage of CpG sites unmethylated; the analytics system identifies such fragments as hypermethylated fragments or hypomethylated fragments. Example thresholds for length of fragments (or CpG sites) include more than 3, 4, 5, 6, 7, 8, 9, 10, etc. Example percentage thresholds of methylation or unmethylation include more than 80%, 85%, 90%, or 95%, or any other percentage within the range of 50%-100%. See, e.g., U.S. patent application Ser. No. 15/931,022, entitled “Model-Based Featurization And Classification,” and filed on May 13, 2020, the content of which is incorporated herein by reference in its entirety.

Method 400 includes, at block 406, predicting a tumor mutational burden (TMB) for a tissue of interest at the subject using a TMB prediction model that receives the feature matrix as input and outputs a predicted TMB. The predicted TMB can be representative of, or otherwise correspond to, an estimated total number of nonsynonymous somatic mutations for the tissue of interest at the subject.

In some examples, the TMB prediction model is a predictive machine learning model trained on samples (e.g., training samples where both tissue data and cfDNA data is available from the same subjects) to predict tissue TMB using cfDNA data. The TMB prediction model can be a regression model trained to predict tissue TMB using a combination of features derived from the sequence data, such as features from plasma SNVs, SCNAs from cfDNA, and/or cfDNA methylation measurements (targeted or across the genome). For instance, the model can be fitted to predict tissue TMB from a combination of blood-derived signals, such as SNVs, SCNAs and/or DNA methylation across the genome or certain genomic regions.

In some exemplary embodiments, the TMB prediction model comprises a statistical model trained with a training set comprising training data obtained from sequencing a plurality of training samples of cfDNA collected from a plurality of subjects. The training data obtained from each training sample can correspond to matched tissue data obtained from a tumoral tissue sample collected from the same subject. The statistical model can comprise a L1 penalized linear regression model. Other types of models can be contemplated, including normal linear regression, L2-penalized linear regression, elastic net, etc. In some examples, performance of the model can be evaluated with k-fold cross-validation, such as a 10-fold cross-validation.

In some examples, the training data is obtained from targeted sequencing of the plurality of cfDNA train samples. In some examples, the matched tissue data is obtained by whole exome sequencing of the corresponding plurality of tumoral tissue samples. In some embodiments, the method includes, for each train sample in the plurality of train samples: labeling the training data with a corresponding ground truth TMB determined from the corresponding matched tissue data, and generating a predicted TMB from the labeled training data using the statistical model. The predicted TMB can be correlated with the corresponding ground truth TMB.

In some cases, samples selected for training the TMB prediction model include samples corresponding to cancer stage III or stage IV conditions, and/or training samples identified as having a TF that exceeds a minimum TF. For instance, the method can include cleaning training data by removing data from samples that do not have a TF greater than and/or equal to a minimum TF of 1%. The TF of a sample can comprise a maximum allele frequency (AF) of all mutations in the sample. In some cases, the minimum TF can depend on a type of sequencing assay utilized for generating the sequence data.

Method 400 includes, at block 408, determining whether a set of criteria has been met, wherein the set of criteria includes at least one criterion that is met when the predicted TMB is high (e.g., when the predicted TMB meets and/or otherwise exceeds a predetermined value). Method 400 includes, at block 410, in accordance with a determination that the set of criteria has been met, determining that the subject is likely to respond to the treatment. Method 400 includes, at block 412, in accordance with a determination that the set of criteria has not been met, determining that the subject is not likely to respond to the treatment, and/or otherwise forgoing the determination that the subject is likely to respond.

As discussed above, tissue TMB can be used to assess whether an JO drug or treatment is appropriate for a cancer patient. In particular, high TMB is associated with improved survival for patients undergoing immunotherapy, and thus predicted high tissue TMB is indicative of a likely responder to treatment. With the present disclosure, predicting TMB from cfDNA for tissue provides a non-invasive technique for using TMB as a clinical biomarker to determine the subject's eligibility for a potential treatment (immunotherapy/IO) or effectiveness of an already administered treatment. Example JO treatments can include anti-PD1 therapy or anti-PDL1 inhibitor. The anti-PD1 therapy can be assessed for eligibility in treating tumors associated with non-small cell lung cancer (NSCLC) or melanoma. Example JO drugs for cancer immunotherapy (CIT) can include, but are not limited to, Atezolizumab, Durvalumab, Ipilimumab, Nivolumab, and/or Pembrolizumab.

In some cases, method 400 further includes administering treatment if the subject is determined to be a likely responder (e.g., based on whether the set of criteria is met), and/or forgoing administering treatment if the subject is not determined to be a likely responder. In some examples, the method 400 further includes continuing administration of the treatment to the subject in accordance with the determination that the subject is likely to respond to the treatment, and/or altering administration of the treatment to the subject in accordance with the determination that the subject is not likely to respond. For instance, continuing administration can include administering the same treatment and/or proceeding with next steps in a course of treatments, while altering administration can include adjusting treatment dosage/type, ceasing treatment, switching to a different treatment, etc.

Additionally and/or alternatively, the set of criteria can include one or more other criterion that can be indicative of whether an JO drug or treatment is appropriate for a cancer patient. As discussed further below, such criterion can correspond to determining whether a predicted TH from cfDNA for tissue is indicative of a likely responder, and/or determining whether a predicted TF from cfDNA is indicative of a likely responder. Any of the TMB, TH, and/or TF, predicted or otherwise estimated from cfDNA, can be utilized alone or in any combination to assess whether a subject is likely to respond to an immunotherapy/IO treatment, and/or otherwise determine whether to administer or continue administering the treatment. In some cases, whether one or more of TMB, TH, and/or TF are assessed can depend on the patient's disease type, cancer type, cancer stage, immunotherapy type being considered, age, and/or other factors that can impact which biomarkers are best suited for predicting the patient's response to a treatment.

Predicting Tumoral Heterogeneity (“TH”) from cfDNA to Determine Treatment Response

In some embodiments, tumoral heterogeneity (TH) can be a predictive biomarker for immuno oncology treatment (TO) response, alone or in combination with TMB. For instance, tumors that respond best to checkpoint inhibitors have high homogeneous mutational burden, whereas tumors that respond poorly to IO therapy have low homogeneous mutational burden. In general, a tumoral tissue sample is considered homogeneous tissue if the tumoral tissue sample has a low level of subclonal mutations. The tumoral tissue sample is heterogeneous tissue if the tumoral tissue sample has a high level of subclonal mutations. Therefore, measurement of TH can be of interest for predicting tumors that will not respond to checkpoint inhibition. Accordingly, the present disclosure provides methods for identifying heterogeneous tumors (or otherwise disambiguating heterogeneous and homogeneous tumors) from targeted panel sequencing of cfDNA.

For instance, referring back to FIG. 4, in some embodiments, method 400 includes, at block 414, determining whether the set of criteria has been met, whereby the set of criteria further includes a criterion that is met when the predicted TMB is high and a tissue tumoral heterogeneity (TH) predicted from cfDNA is indicative of a homogeneous tissue. For example, the method 400 can include determining whether the predicted TMB is high, and if so, further predicting, based on the sequence data, the TH for the tissue of interest. Additionally or alternatively, the TH can be predicted prior to determination of the predicted TMB and/or concurrently therewith. Further, in some examples, method 400 includes determining whether the predicted TH is indicative of homogeneous or heterogeneous tissue, and in accordance with a determination that the predicted TH is indicative of the homogeneous tissue (e.g., high homogeneity or low heterogeneity), determining that the subject is likely to respond to the treatment, whereas in accordance with a determination that the predicted TH is indicative of the heterogeneous tissue (e.g., low homogeneity or high heterogeneity), determining that the subject is not likely (e.g., or otherwise less likely) to respond to the treatment. In some cases, method 400 can include, subsequent to the determination that the predicted TMB is not high, forgoing determining whether the predicted TMB corresponds to a homogeneous or heterogeneous sample, and/or determining that the subject is not responsive to the treatment.

In some examples, predicting the TH from cfDNA data utilizes a TH prediction model. The TH prediction model can be a statistical model, such as a linear regression learning model (e.g., L1 or L2-regularized model or non-regularized model) trained to predict heterogeneity based on cfDNA data. For example, the model can be trained using paired tumor-cfDNA samples, with each paired sample having a heterogeneity score that describes the fraction of mutations present in both tumor and cfDNA. The TH prediction model can recapitulate TH determined from the paired tumor-cfDNA sequencing. In some exemplary embodiments, the TH prediction model is trained on a training set comprising a plurality of training samples that are derived from cfDNA samples having matched tissue data from tumoral tissue samples, whereby training samples having high cfDNA-tissue concordance correspond to low coefficient of variation (low CV) of cfDNA variant allele frequencies and are homogeneous, and training samples having low cfDNA-tissue concordance correspond to high coefficient of variation (high CV) of cfDNA variant allele frequencies and are heterogeneous. It is noted that concordance can represent an amount of matched variants compared to an amount of total variants in both tumor and cfDNA samples from a subject, such that high cfDNA-tissue concordance indicates a high amount of overlap between the samples, and low cfDNA-tissue concordance indicates a lower amount of overlap between the samples. The coefficient of variation (CV) can be a standard deviation of the allele frequency of SNV calls divided by the mean allele frequency of cfDNA variants.

To generate the predicted TH (and/or probabilities thereof), the TH prediction model can analyze a set of features in the sequence data. The set of features can include one or more of an allele frequency (AF) of single nucleotide variant (SNV) calls in the cfDNA sample, a mean allele frequency of cfDNA variants in the cfDNA sample, a ratio of minimum to maximum allele frequency of cfDNA variants in the cfDNA sample, and a reciprocal fraction of a number of cfDNA variants in the cfDNA sample. In some examples, the set of features can include copy number aberration (CNA) profiles and/or methylation-related features/status (e.g., CpG based analysis). In some cases, the set of features can be included in the feature matrix generated at step 404. Alternatively, the feature matrix can be generated separately, and/or subsequent to a determination that the TMB is high.

In some exemplary embodiments, the TH prediction model is a linear regression model that determines a coefficient of variation (CV) of the allele frequency of SNV calls based on the set of features. As noted above, the coefficient of variation (CV) can be a standard deviation of the allele frequency of SNV calls divided by the mean allele frequency of cfDNA variants. In accordance with a determination that the CV is low, the TH prediction model can determine that the predicted TH is indicative of homogeneous tissue, and in accordance with a determination that the CV is high, the TH prediction model can determine that the predicted TH is indicative of heterogeneous tissue. In some examples, the TH prediction model determines a TH score and/or a calculated CV of the sample. In such cases, the determined TH score and/or the calculated CV can be compared to a predetermined TH score and/or a threshold CV to determine whether the cfDNA data is indicative of a low or high homogeneity tissue.

Predicting Tumor Fraction (“TF”) from cfDNA to Determine Treatment Response

Tumor fraction (TF) can be predictive of patient response to immunotherapy and can be used in any combination with TMB, TH, and/or other predictive biomarkers such as methylation score. Accordingly, the present disclosure provides a non-invasive method that associates TF in cfDNA as an indicator of biology and response, as opposed to other methods that take measurements from tumoral tissue directly. In some aspects, measuring TF from cfDNA can allow for prediction with lower evidence or sequencing depths. In some cases, TF is used as a confidence factor in blood based TMB measurements, because variant calls can become more accurate at higher TF. Various methods for determining tumor fraction can be found in International Patent Application No. PCT/US2019/027756, entitled “Systems and Methods for Determining Tumor Fraction in Cell-Free Nucleic Acid,” and filed on Apr. 16, 2019, the content of which is incorporated herein by reference in its entirety.

Referring again to FIG. 4, in some embodiments, method 400 includes, at block 116, that the set of criteria further includes a criterion that is met when the predicted TMB is high and a TF computed based on the sequence data corresponds to a positive treatment response. In some cases, whether a computed high or low TF is indicative of treatment response further depends on a type of disease state (e.g., a clinical stage, type of cancer). For instance, the computed TF is indicative of a positive treatment response (e.g., more likely to respond or otherwise have greater benefit from CIT) when the computed TF is a low TF (e.g., <1%, <0.05%) and the disease state is stage IV lung cancer. In some cases, the computed TF is indicative of a positive treatment response when the computed TF is a high TF (e.g., >=1%, >=0.05%) and the disease state is stage III lung cancer. The computed TF can be compared to a threshold TF value or score to determine whether the computed TF is low or high. The threshold TF value or score can depend on a sequencing method or panel used for generating the cfDNA data, or vary for different cancer types or stages being assessed.

Additionally and/or alternatively, in some cases, whether a computed high or low TF is indicative of treatment response further depends on a treatment type (e.g., CIT, or treatment). For instance, in some cases, the computed TF is indicative of a positive treatment response (i.e., more likely to respond or otherwise have greater benefit from treatment) when the computed TF is a low TF (e.g., <1%, <0.05%) and the treatment is a treatment other than cancer immunotherapy (CIT), for both stage III and stage IV lung cancer patients. On the other hand, in some cases, the computed TF is indicative of a negative treatment response (e.g., less likely to benefit from CIT) when the computed TF is low and the treatment is CIT (e.g., and/or the disease state is stage III lung cancer).

Merely by way of example, in some embodiments, the set of criteria further includes a criterion that is met when a tumor fraction (TF) computed based on the sequence data is low. In some cases, the criterion is met when both the predicted TMB is high and the computed TF is low. For example, method 400 can include, subsequent to the determination that the predicted TMB is high, determining whether the TF is low, wherein the TF comprises a fraction of tumor-derived cfDNA over a total amount of cfDNA in the cfDNA sample. The method 400 can include, in accordance with a determination that the TF is low, determining that the subject is likely to respond to the treatment, while in accordance with a determination that the TF is not low, determining that the subject is not likely to respond to the treatment.

In some cases, a higher computed TF is indicative of a more likely responder. For instance, in some examples, the set of criteria further includes a criterion that is met when a tumor fraction (TF) computed based on the sequence data is high. In some cases, the criterion is met when both the predicted TMB is high and the computed TF is high. For instance, as mentioned previously, in some applications, the computed TF can be used as a confidence factor in blood based TMB measurements, because variant calls can become more accurate at higher TF. It is noted that whether a computed high or low TF is indicative of a likely or unlikely treatment responder can depend on how the TF is calculated.

Variations of the present embodiments can be contemplated. For instance, in some examples, a 3-model aggregate weighs TMB, TH, and TF scores estimated from a cfDNA sample and computes a final likelihood for CIT response/benefit. In some examples, additional models accounting for other predictive biomarkers that can be inferred from signals in the cfDNA can be incorporated with the present embodiments for predicting treatment response.

Example Processing System for Predicting and Monitoring Treatment Response

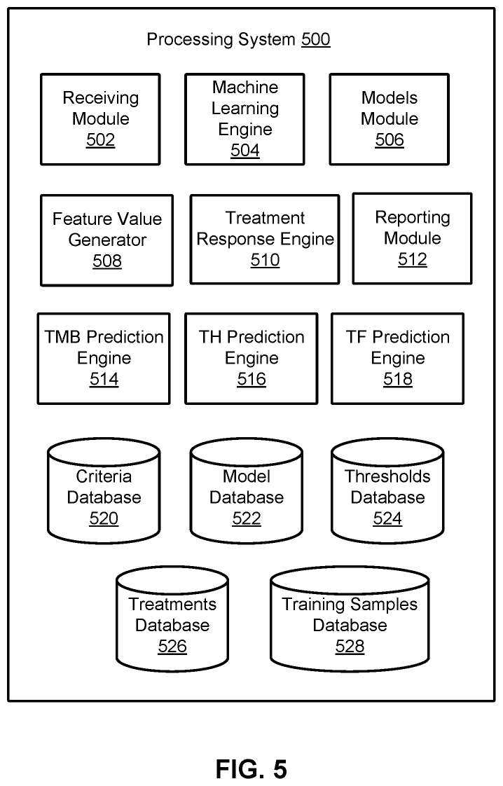

Turning now to FIG. 5, FIG. 5 is a schematic diagram of a processing system 500 for predicting and monitoring treatment response using TMB, TH, and/or TF as predictive biomarkers, according to various embodiments. It is noted that the processing system 500 can include additional components not shown in FIG. 5, such as any of the components of system 200 at FIG. 2, and/or be in operative communication with system 200 (e.g., to receive sequence data/reads and/or variant calls from system 200). As shown at FIG. 5, system 500 includes components that enable the system 500 to perform the steps described at FIG. 4. Such components include a receiving module 502, a machine learning engine 504, a models module 506, a feature value generator 508, a treatment response engine 510, a reporting module 512, a TMB prediction engine 514, a TH prediction engine 516, a TF prediction engine 518, a criteria database 520, a model database 522, a thresholds database 524, a treatments database 526, and a training samples database 528. It is noted that some components can be optional, and multiple components can be combined as a single component.

In some examples, the receiving module 502 can receive sequence data gathered from sequencing the cfDNA sample. For example, the receiving module 502 can receive sequence data, such as sequence reads and/or variant calls, from processing system 200 of FIG. 2. Based on the received sequencing data, the feature value generator 508 can generate a feature matrix that includes feature values corresponding to synonymous mutations, nonsynonymous mutations, AF of variants, sum of the AFs, maximum AFs, and/or other features in the sequence data. The feature matrix can be input into the TMB prediction engine 514 that predicts a tumor mutational burden (TMB) for a tissue of interest at the subject. The TMB prediction engine 514 can implement a TMB prediction model provided by the models module 506 and/or stored in the model database 522 to generate the TMB prediction. The predicted TMB can be assessed by the treatment response engine 510 to determine whether the subject is likely to respond to a certain cancer treatment, which can be stored in the treatments database 526. The treatment response engine 510 utilizes a set of criteria stored at criteria database 520, which can include at least one criterion that is met when the predicted TMB is high. In some examples, the predicted TMB is determined to be high based on a threshold TMB that is stored, for example, in the thresholds database 524. Reporting module 512 can output metrics and results of the treatment response analysis, such as the predicted TMB (and/or TH and TF), a predicted likelihood of treatment response, and/or a recommended treatment plan. The reporting module 512 can be in operative communication with external devices, networks, or user interfaces configured to receive outputs of the analysis.

In some examples, the treatments database 526 includes various immunotherapies and targeted therapeutics, such as various types of PD-1 inhibition, PD-L1 inhibition, or CTL-4 inhibition. PD-1 inhibition targets the programmed death receptor on T-cells and other immune cells. Examples of PD-1 inhibition immunotherapies include Pembrolizumab; Keytruda; Nivolumab; Opdivo; Cemiplimab; Libtayo. PD-L1 inhibition targets the programmed death receptor ligand expressed by tumor and regulatory immune cells. Examples of PD-L1 Inhibition immunotherapies include Atezolizumab; Tecentriq; Avelumab; Bavencio; Durvalumab; Imfinzi. CTL-4 inhibition targets T-cell activation. Examples of CTL-4 inhibition immunotherapies include Ipilimumab; Yervoy.

In some examples, the treatments database 526 includes data associated with known cancer immunotherapy (CIT) drugs, such as any of the following drugs: Atezolizumab, Durvalumab, Ipilimumab, Nivolumab, Pembrolizumab. In some cases, the treatments database 526 stores information on certain immunotherapies and targeted therapeutics, such as an immunoglobulin, a protein, a peptide, a small molecule, a nanoparticle, or a nucleic acid. In some embodiments, the therapies comprise an antibody, or a functional fragment thereof. In some embodiments, the antibody is selected from the group consisting of: Rituxan® (rituximab), Herceptin® (trastuzumab), Erbitux® (cetuximab), Vectibix® (Panitumumab), Arzerra® (Ofatumumab), Benlysta® (belimumab), Yervoy® (ipilimumab), Perjeta® (Pertuzumab), Tremelimumab®, Opdivo® (nivolumab), Dacetuzumab®, Urelumab®, Tecentriq® (atezolizumab, MPDL3280A), Lambrolizumab®, Blinatumomab®, CT-011, Keytruda® (pembrolizumab, MK-3475), BMS-936559, MED14736, MSB0010718C, Imfinzi® (durvalumab), Bavencio® (avelumab) and margetuximab (MGAH22).

In some examples, the treatments database 526 maps certain treatments to certain cancer types and/or certain variants that may be detected during sequence processing. For example, the anti-PD1 therapy is assessed for eligibility in treating tumors associated with non-small cell lung cancer (NSCLC) or melanoma. For non-small cell lung cancer indications, variants or mutations that can be biomarkers for immunotherapy treatments can include EGFR exon 19 deletions & EGFR exon 21 L858R alterations (e.g., for therapies such as Gilotrif® (afatinib), Iressa® (gefitinib), Tagrisso® (osimertinib), or Tarceva® (erlotinib)); EGFR exon 20 T790M alterations (e.g., Tagrisso® (osimertinib)); ALK rearrangements (e.g., Alecensa® (alectinib), Xalkori® (crizotinib), or Zykadia® (ceritinib)); BRAF V600E (e.g., Tafinlar® (dabrafenib) in combination with Mekinist® (trametinib)); single nucleotide variants (SNVs) and indels that lead to MET exon 14 skipping (e.g., Tabrecta™ (capmatinib)).

For melanoma indications, variants or mutations that can be biomarkers for immunotherapy treatments can include BRAF V600E (e.g., Tafinlar® (dabrafenib) or Zelboraf® (vemurafenib)); BRAF V600E or V600K (e.g., Mekinist® (trametinib) or Cotellic® (cobimetinib), in combination with Zelboraf® (vemurafenib)).

For breast cancer indications, variants or mutations that can be biomarkers for immunotherapy treatments can include ERBB2 (HER2) amplification (e.g., Herceptin® (trastuzumab), Kadcyla® (ado-trastuzumab-emtansine), or Perjeta® (pertuzumab)); PIK3CA alterations (e.g., Piqray® (alpelisib)).

For colorectal cancer indications, variants or mutations that can be biomarkers for immunotherapy treatments can include KRAS wild-type (absence of mutations in codons 12 and 13) (e.g., Erbitux® (cetuximab)); KRAS wild-type (absence of mutations in exons 2, 3, and 4) and NRAS wild type (absence of mutations in exons 2, 3, and 4) (e.g., Vectibix® (panitumumab)).

For ovarian cancer indications, variants or mutations that can be biomarkers for immunotherapy treatments can include BRCA1/2 alterations (e.g., Lynparza® (olaparib) or Rubraca® (rucaparib)).

For prostate cancer indications, variants or mutations that can be biomarkers for immunotherapy treatments can include Homologous Recombination Repair (HRR) gene (BRCA1, BRCA2, ATM, BARD1, BRIP1, CDK12, CHEK1, CHEK2, FANCL, PALB2, RAD51B, RAD51C, RAD51D and RAD54L) alterations (e.g., Lynparza® (olaparib)).

For solid tumor cancer indications, variants or mutations that can be biomarkers for immunotherapy treatments can include a tumor mutational burden (TMB) that is greater than or equal to 10 mutations per megabase (e.g., Keytruda® (pembrolizumab)).

Referring back to FIG. 5, the models module 506 and/or model database 522 can store and/or implement the TMB prediction model, which can comprise a statistical model trained with a training set comprising train data obtained from sequencing a plurality of train samples of cfDNA collected from a plurality of subjects. The statistical model can be trained by the machine learning engine 504 using train data stored at the training samples database 528. The train data obtained from each train sample can correspond to matched tissue data obtained from a tumoral tissue sample collected from the same subject, and the matched tissue data can also be stored at the training samples database 528. To train the statistical model, the machine learning engine 504 can, for each train sample in the plurality of train samples, label the train data with a corresponding ground truth TMB determined from the corresponding matched tissue data which can be retrieved from the training samples database 528, generate a predicted TMB from the labeled train data using the statistical model, and correlate the predicted TMB with the corresponding ground truth TMB.

As further shown in FIG. 5, the processing system 500 includes the TH prediction engine 516, which can predict the TH based on the sequence data and determine whether the predicted TH is indicative of homogeneous or heterogeneous tissue. With the predicted TH and/or the homogeneous/heterogeneous tissue type, the treatment response engine 510 can determine whether the subject is likely to respond to the treatment. For instance, the treatment response engine 510 can determine that the subject is likely to respond to the treatment if the predicted TH is indicative of the homogeneous tissue. In some cases, the treatment response engine 510 can make the determination based on a criterion stored in the criteria database 520, such as determining whether a criterion has been met, whereby the criterion requires when the predicted TMB is high and the predicted TH is indicative of a homogeneous tissue.

In some examples, the models module 506 and/or model database 522 includes a TH prediction model. The TH prediction model can be used by the TH prediction engine 516 to receive a set of features in the sequence data as input and output the predicted TH. The set of features can be generated by the feature value generator 508 and can include at least one feature corresponding to one or more of: an allele frequency of single nucleotide variant (SNV) calls in the cfDNA sample, a mean allele frequency of cfDNA variants in the cfDNA sample, a ratio of minimum to maximum allele frequency of cfDNA variants in the cfDNA sample, a reciprocal fraction of a number of cfDNA variants in the cfDNA sample, copy number aberration (CNA) profiles, and/or methylation-related features/status based on a CpG analysis.

In some examples, the TH prediction model is a linear regression model. The linear regression model can be L1 or L2 regularized. In an exemplary embodiment, the linear regression model is non-regularized. The TH prediction engine 516 can determine a coefficient of variation of the allele frequency of SNV calls based on the set of features, and if the coefficient of variation is low, determine that the predicted TH is indicative of homogeneous tissue, or if the coefficient of variation is high, determine that the predicted TH is indicative of heterogeneous tissue. In some cases, the TH prediction engine 516 and/or the feature value generator 508 can calculate the coefficient of variation as a standard deviation of the allele frequency of SNV calls divided by the mean allele frequency of cfDNA variants. In some examples, the TH prediction model generates a TH score, and if the score is greater than a predetermined threshold score (e.g., a threshold score retrieved from the thresholds database 524), determine that the predicted TH is indicative of a heterogeneous tissue.

In some examples, the TH prediction model is a statistical model provided by the models database 522, which stores the TH prediction model, and/or provided by the models module 506 which can retrieve and/or implement the TH prediction model along with the TH prediction engine 516. The statistical model can be trained (e.g., by the machine learning engine 504) on a training set of cfDNA samples having matched tissue data from tumoral tissue samples. Such training sets and data can be stored in the training samples database 528. In some examples, the training samples having high cfDNA-tissue concordance correspond to low coefficient of variation of cfDNA variant allele frequencies and are homogeneous, and the training samples having low cfDNA-tissue concordance correspond to high coefficient of variation of cfDNA variant allele frequencies and are heterogeneous. As noted above, the concordance can refer to a number of matched variants divided by a total number of variants in both cfDNA and its tissue samples.

As shown in FIG. 5, the system 500 includes the TF prediction engine 518 which can determine whether the TF is high or low. For example, the criteria database 520 can include a criterion that is met when the predicted TMB is high and a tumor fraction (TF) computed based on the sequence data is low. The TF prediction engine 518 can compute the TF as a fraction of tumor-derived cfDNA over a total amount of cfDNA in the cfDNA sample. The treatment response engine 510 can determine based on a low TF that the subject is likely to respond to the treatment, or based on a higher TF that the subject is not likely to respond to the treatment. Such results can be reported or otherwise prepared for output by the reporting module 512.

In some cases, the treatment response engine 510 utilizes a 3-model aggregate provided by the models module 506 and/or model database 522 to determine, based on the computed TMB, TH, and TF assessments, a final likelihood for treatment response. For example, the 3-model aggregate can weigh the TMB, TH, and TF scores. In some examples, weighting values can depend on cancer type or stage, the patient's age, gender, or other factors.

Example TMB Prediction 1: Using Stages III and IV Cancers

Tissue TMB is a clinical biomarker for immuno oncology therapies and is currently utilized to determine eligibility for anti-PD1 therapy, which can treat melanoma and non-small cell lung cancers. An objective of this investigation was to develop a model to predict tissue TMB based on cfDNA data from the Cell-Free Genome Atlas Study (CCGA).

CCGA [NCT02889978] is a prospective, multi-center, case-control, observational study with longitudinal follow-up. The study enrolled 9,977 of 15,000 demographically-balanced participants at 141 sites. Blood was collected from subjects with newly diagnosed therapy-naive cancer (C, case) and participants without a diagnosis of cancer (noncancer [NC], control) as defined at enrollment. This preplanned substudy included 1628 cases and 1172 controls, across twenty tumor types and all clinical stages. Samples were divided into training (1,785) and test (1,015) sets prior to analysis. Samples were selected to ensure a prespecified distribution of cancer types and non-cancers across sites in each cohort, and cancer and non-cancer samples were frequency age-matched by gender.

Cell-free DNA was isolated from plasma, while genomic DNA (gDNA) was isolated from white blood cells (WBCs) and tumor tissue using standard methodologies. Three distinct high-intensity sequencing approaches were employed in cfDNA analysis: (i) cfDNA whole-genome bisulfite sequencing (WGBS; 30× depth) in which normalized scores were generated using abnormally methylated fragments, (ii) paired cfDNA and WBC whole-genome sequencing (WGS; 30× depth) in which a novel machine learning algorithm generated cancer-related signal scores and joint analysis identified shared events, and (iii) paired cfDNA and WBC targeted sequencing (507-gene panel; 60,000× depth, referred to herein as the “ART” assay) in which a joint caller removed WBC-derived somatic variants and residual technical noise. WBC gDNA was subjected to targeted sequencing to identify clonal hematopoiesis (CH). Tumor tissue gDNA was subjected to WGS to identify somatic variants, which were used to calculate cfDNA tumor fraction. Additional details of the CCGA study can be found in International Patent Application No. PCT/US2019/027756, entitled “Systems and Methods for Determining Tumor Fraction in Cell-Free Nucleic Acid,” and filed on Apr. 16, 2019, the content of which is incorporated herein by reference in its entirety.

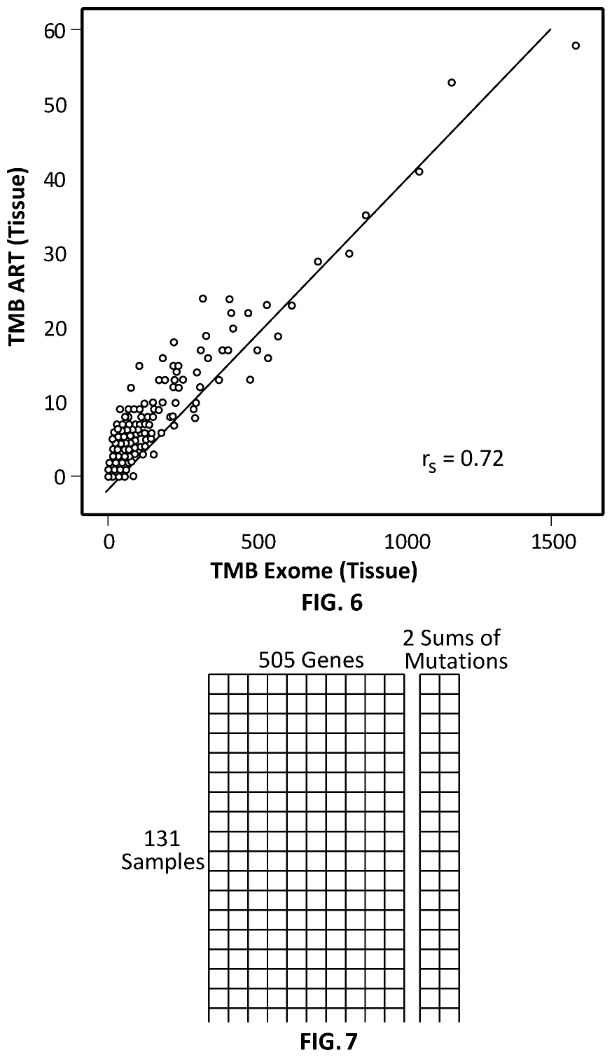

In this present investigation, the TMB is defined as the total number of nonsynonymous point mutations for a sample. In this example, the total number of nonsynonymous point mutations included indels. Typically, TMB is generated by whole-exome sequencing of tissue data. The plot at FIG. 6 shows that the TMB for whole-exome sequenced regions of the tissue data from this investigation (x-axis) is correlated with the TMB computed from only ART regions of the exome data (y-axis), with a Spearman correlation coefficient at 0.72. The ART regions were included in the ART panel discussed above in the CCGA study.

An estimate model to predict tissue TMB from the cfDNA ART data was designed, where dependent variable “y” corresponds to tissue TMB from the ART regions used to supervise linear regression, and independent “X” corresponds to features from the cfDNA ART data. The goal was to train a model that predicts the TMB from blood-based cfDNA data, such that in the absence of tissue data, the model can predict tissue TMB from a blood sample. The predicted tissue TMB can then be used as a non-invasive biomarker for IO treatment.

FIG. 7 illustrates a diagram of a feature matrix derived from the cfDNA ART data that was used to train the model. The model was trained on samples having tissue data, and more specifically, 131 samples consisting of stage III and stage IV samples with a TF>0.001. As shown in FIG. 7, the features in the matrix included: a number of nonsynonymous somatic mutations for each gene at each sample position, a total number of somatic mutations for each sample, and a total number of nonsynonymous somatic mutations for each sample. Here, restricting the training data to stage III and stage IV samples and further using TF to filter the data reduced noise in the data. It is noted that other approaches can be used for filtering the training data, such as limiting the training data to only top cancer types that have a large amount of mutations, and/or setting the TF filter to a higher TF threshold (e.g., 1% or more).

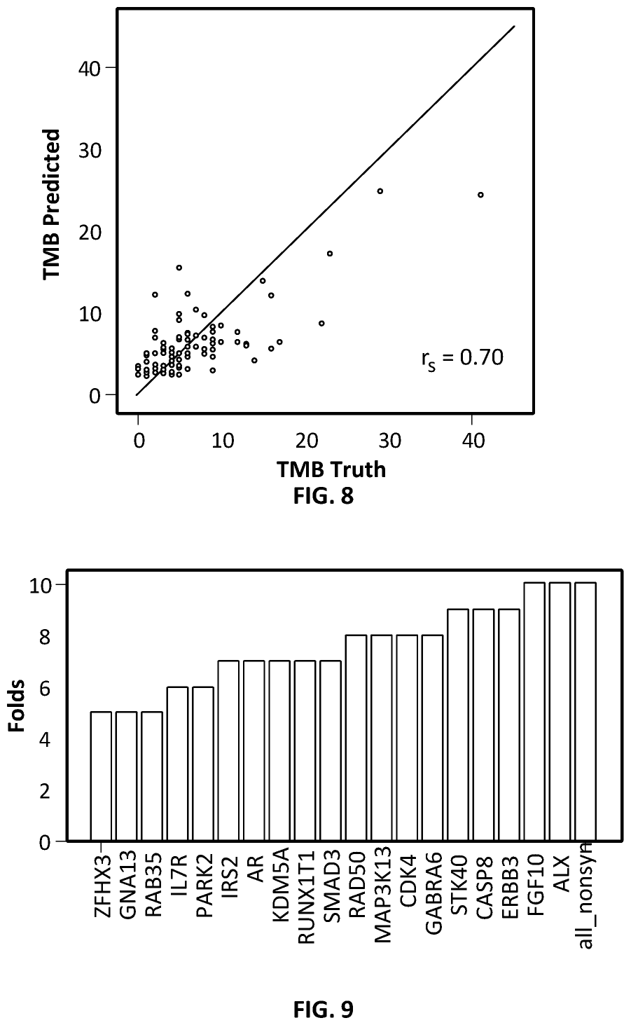

A model was fitted using L1-penalized linear regression to generate a TMB prediction model. As shown at FIG. 8, the predicted TMB values (y-axis) were correlated to the original ground truth values (x-axis) with a Spearman correlation coefficient of 0.70. Further, the L1-penalized regression provided insight into consistent predictors of TMB because features indicated by non-zero regression coefficients were selected as important features. For instance, FIG. 9 illustrates recurring features across the folds of the 10-fold cross validation. As demonstrated at FIG. 9, FGF10, ALK, and using the total sum of nonsynonymous mutations of a sample were consistent predictors of TMB across all of the cross validation folds. On the other hand, gene features for STK40, CASP8, and ERBB3 were present across only 9 of the 10 cross-validation folds and therefore may be considered somewhat less important for predicting TMB.

In summary, in this investigation, a model was trained based on cfDNA ART data to predict TMB using TMB derived from tissue data. The training data included somatic nonsynonymous mutations from stage III and IV samples with TF>0.001. The predicted TMB from cfDNA was correlated with the ground truth TMB from the tissue data. It is further contemplated that a variety of TMB prediction models can be generated and trained, such as a cancer type specific modeling where each model for predicting TMB is specific to a cancer type.

Example TMB Prediction 2: Using Cancers with High Number of Mutations

A second investigation predicted tissue ART TMB using cancers with a high number of mutations. Here, a model was trained on 103 samples consisting of colorectal, esophageal, head/neck, hepatobiliary, lung, lymphoma, multiple myeloma, ovarian, and pancreas cancer types, with a TF>0.001. A feature matrix was derived from the cfDNA ART data and included the same features as those discussed above for the first TMB prediction investigation.

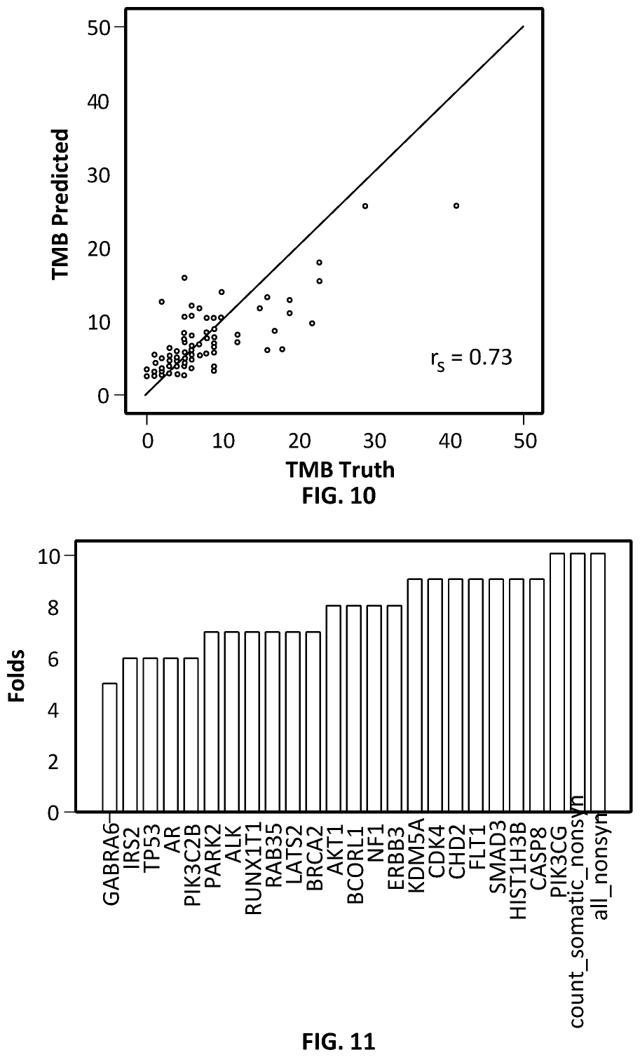

A model was fitted using L1-penalized linear regression and 10-fold cross validation. As shown at FIG. 10, the predicted TMB values (y-axis) are correlated to the original ground truth values (x-axis), with a Spearman correlation coefficient of 0.73. FIG. 11 illustrates recurring features across the folds of the 10-fold cross validation, as identified by the L1-penalization process. As demonstrated at FIG. 11, consistent predictors of TMB across all of the cross validation folds included PIK3CG, all non-synonymous mutations for a sample, and all somatic mutations for the sample.

Example TH Prediction

Tumor heterogeneity is predictive of IO response and can be combined with TMB as a predictive biomarker. This investigation was directed to training a predictive model for TH that relies on allele frequencies of SNV calls in cfDNA data. Training was performed with cfDNA samples that had matched tissue data from the CCGA study described above.

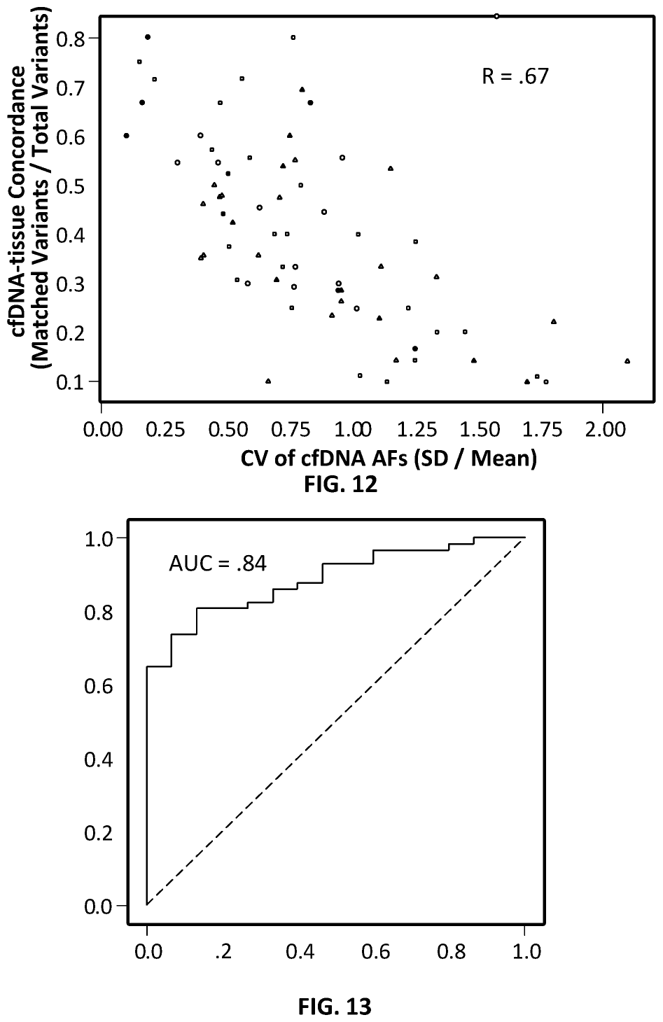

FIG. 12 is a plot showing cfDNA-tissue concordance (defined as matched variants/total variants; y-axis) plotted against the coefficient of variation (CV) of cfDNA allele frequencies (AFs) (defined as standard deviation/mean; x-axis). With a correlation coefficient of 0.67, this plot illustrates that the variability in allele frequencies of cfDNA can be predictive of cfDNA-tissue concordance. Here, the cfDNA-tissue concordance is calculated as a fraction of all cfDNA and tissue variant calls identified in both cell-free and tissue sample types, and uses filtered Sentieon tissue variant calls. In FIG. 12, samples high on cfDNA-tissue concordance (y-axis) have strong agreement between mutations identified in the cfDNA and tissue samples, suggesting that such tumors are homogeneous. On the other hand, samples low on the y-axis had low concordance, suggesting that a number of mutations in the cfDNA sample were not found in the corresponding tissue sample, and vice versa. On the x-axis, samples closer to the y-axis have a lower range of AFs in the tumor, while samples further from the y-axis have a higher range of AFs. Accordingly, this plot illustrates that as variability increases along the x-axis, homogeneity decreases along the y-axis, suggesting that cfDNA data can be used to obtain information about the agreement between cfDNA and tissue data, which can be predictive of homogeneity in the tumor, which further can serve as a predictive biomarker for 10 response.

A linear model was trained on the CCGA-1 samples with matched tissue samples to distinguish between homogeneous and heterogeneous samples having high TMB. Various features that quantified the distribution of allele frequencies of variants were tested, and a final list of features used included: mean AF of variants, min/max AF of variants, CV of AF of variants, and 1/(number of variants). These final features were the most predictive for the model, with the CV of AF of variants considered the most predictive feature among the set (see, e.g., FIG. 12 above). The training included linear regression and 10-fold cross validation.

FIG. 13 demonstrates the performance of the trained model in predicting low concordance samples among the high TMB samples. Specifically, the ROC curve captures samples having more than 6 variants in the cfDNA and was evaluated for classification of low-concordance samples having a cfDNA-tissue concordance greater than 0.25. With an area under the curve (AUC) at 0.84, the ROC curve indicates that the model is useful in identifying samples that have high TMB and low concordance, and that such predictions can be performed based on cfDNA data only. Such samples with high TMB and low concordance are unlikely to respond to IO therapy.

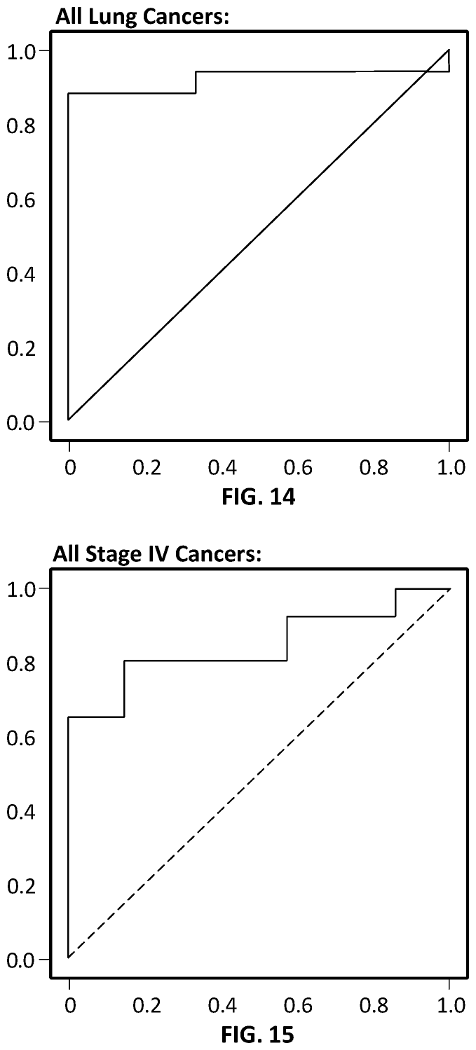

FIG. 14 shows an ROC curve that demonstrates the performance of the trained model on all lung cancers. FIG. 15 shows an ROC curve that demonstrates the performance of the trained model across all stage IV cancers. Performance of the model in FIGS. 14 and 15 is similar to the performance demonstrated at FIG. 13.

In summary, this investigation showed that a linear regression model trained on cfDNA recapitulates TH measured from a cfDNA-tumor comparison. It is further noted that such TH predictive models can be trained by other manners, such as training based on samples of patients that responded to therapy and patients that did not respond to therapy. Such trained models can provide useful insight into therapy selection.

Survival Probabilities with CIT

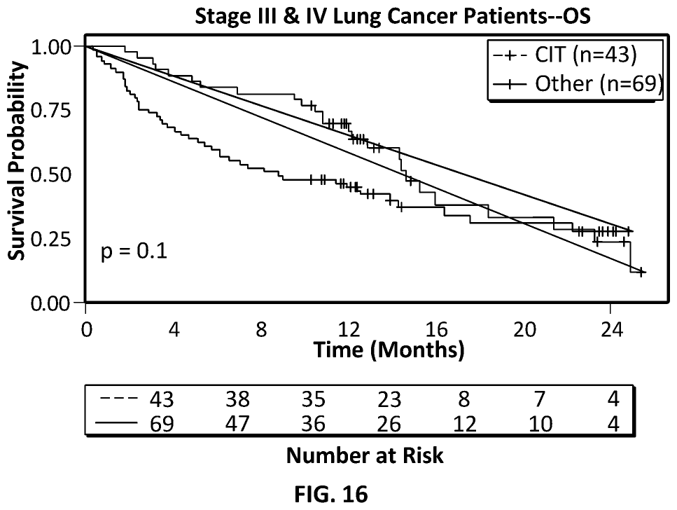

FIGS. 16-25 demonstrate overall survival probabilities for CCGA-1 patients treated with CIT (cancer immunotherapy) compared to other types of treatments. In this investigation, the CIT patients were treated with any of the following drugs: Atezolizumab, Durvalumab, Ipilimumab, Nivolumab, and Pembrolizumab. Table 1 shows the cancer stage and type of patients treated with CIT, and Table 2 shows the cancer stage and type of patients treated with a treatment other than CIT.

TABLE 1

| | I | II | III | IV |
|---|---|---|---|---|
| Bladder | 0 | 0 | 0 | 2 |
| Breast | 0 | 1 | 2 | 0 |
| Cervical | 0 | 0 | 1 | 0 |
| Esophageal | 0 | 0 | 0 | 1 |
| Head/Neck | 0 | 0 | 0 | 1 |
| Hepatobiliary | 1 | 0 | 0 | 4 |
| Lung | 1 | 4 | 13 | 30 |
| Lymphoma | 0 | 1 | 1 | 0 |
| Melanoma | 0 | 2 | 3 | 3 |
| Other | 0 | 1 | 0 | 0 |
| Renal | 0 | 0 | 1 | 2 |
| Two primaries | 0 | 0 | 0 | 1 |
| Unknown | 0 | 0 | 0 | 1 |

TABLE 2

| | I | II | III | IV | Leukemia |
|---|---|---|---|---|---|
| Anorectal | 1 | 1 | 5 | 1 | 0 |
| Bladder | 2 | 3 | 1 | 3 | 0 |
| Breast | 221 | 169 | 62 | 10 | 0 |
| Cervical | 7 | 3 | 3 | 3 | 0 |
| Colorectal | 2 | 11 | 26 | 25 | 0 |
| Esophageal | 2 | 12 | 8 | 4 | 0 |
| Gastric | 3 | 10 | 3 | 6 | 0 |
| Head/Neck | 0 | 2 | 4 | 11 | 0 |
| Hepatobiliary | 3 | 3 | 2 | 11 | 0 |
| Leukemia | 0 | 0 | 0 | 0 | 14 |
| Lung | 10 | 16 | 35 | 52 | 0 |
| Lymphoma | 6 | 12 | 9 | 11 | 0 |
| Melanoma | 0 | 2 | 3 | 3 | 0 |
| MM | 7 | 3 | 7 | 0 | 0 |
| Other | 0 | 3 | 2 | 4 | 0 |
| Ovarian | 0 | 0 | 12 | 9 | 0 |
| Pancreas | 5 | 8 | 2 | 25 | 0 |
| Prostrate | 1 | 10 | 1 | 6 | 0 |
| Renal | 0 | 1 | 2 | 6 | 0 |
| Thyroid | 0 | 0 | 0 | 1 | 0 |
| Two primaries | 1 | 0 | 1 | 1 | 0 |
| Unknown | 0 | 0 | 2 | 11 | 0 |
| Uterine | 6 | 1 | 1 | 3 | 0 |

FIG. 16 shows the overall survival of stage III and IV lung cancer patients that were treated with CIT versus other treatments. As demonstrated in the graph, the lung cancer patients treated with CIT (n=43) had a higher survival probability than those treated with other treatments (n=69) over a 24 month timeframe.

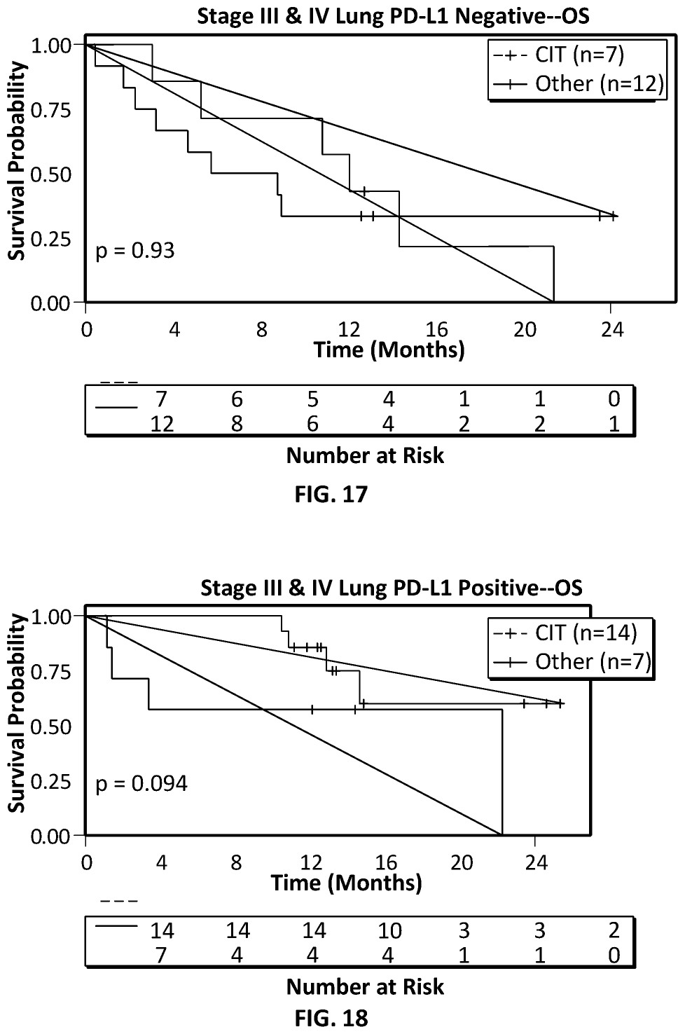

FIG. 17 illustrates the use of PD-L1 negative expression as a biomarker for CIT benefit for stage III and IV lung cancer patients treated with CIT (n=7) versus other treatments (n=12). FIG. 18 illustrates the use of PD-L1 positive expression as a biomarker for CIT benefit for stage III and IV lung cancer patients treated with CIT (n=14) versus other treatments (n=7). In both figures, the charts show that patients treated with CIT generally have greater survival probability over a period of time than those treated with other treatments.

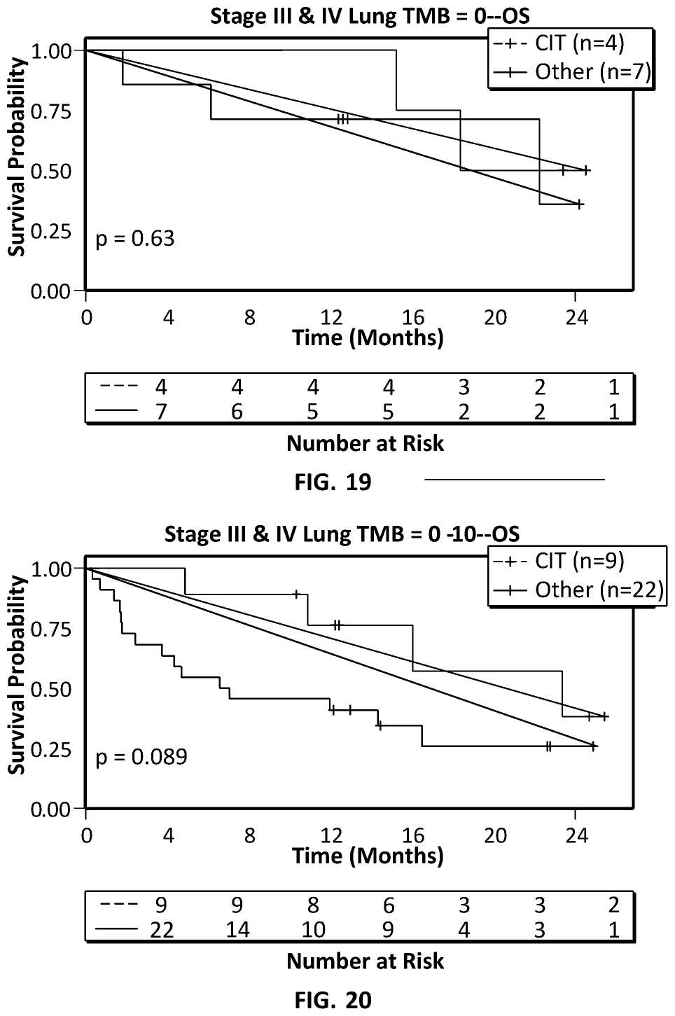

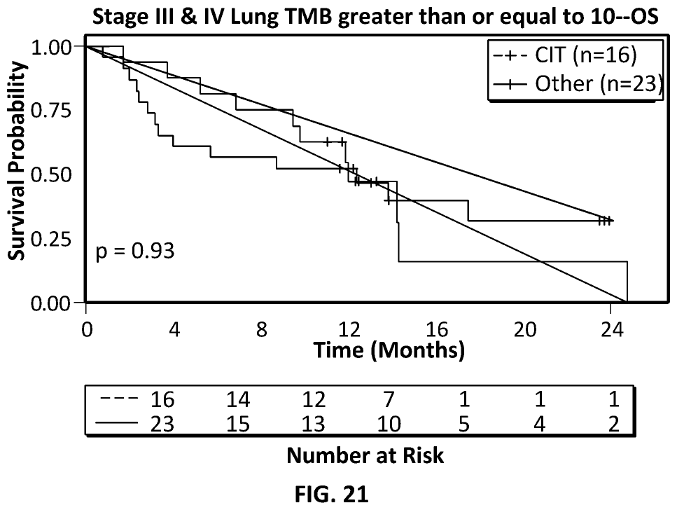

FIGS. 19-21 demonstrate using TMB as a biomarker for CIT benefit for stage III and IV lung cancer patients. In particular, FIG. 19 illustrates stage III and IV lung cancer patients treated with CIT (n=4) versus other treatments (n=7), where the patients had a TMB=0. FIG. 20 illustrates stage III and IV lung cancer patients treated with CIT (n=16) versus other treatments (n=23), where the patients had a TMB between 0 and 10. FIG. 21 illustrates stage III and IV lung cancer patients treated with CIT (n=9) versus other treatments (n=22), where the patients had a TMB greater than or equal to 10. As shown across FIGS. 19-21, patients treated with CIT generally had greater survival probability over a period of time than those treated with other treatments. The difference in benefit is most pronounced in FIG. 21 for patients with higher TMB (TMB greater than or equal to 10).

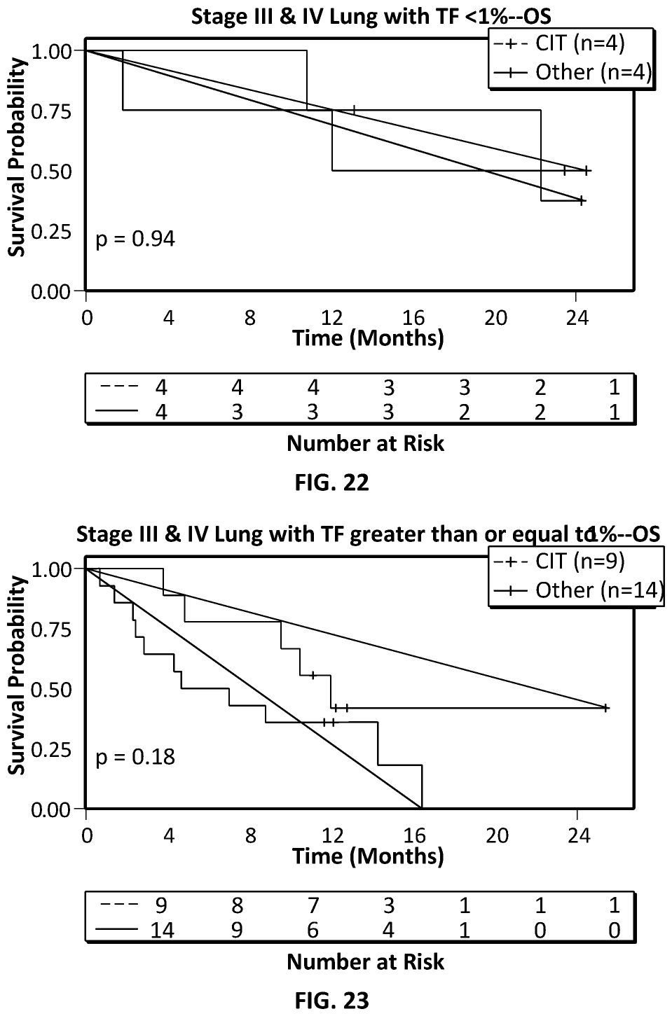

FIGS. 22-23 show data demonstrating the use of TF as a biomarker for CIT response for stage III and IV lung cancer patients. In particular, FIG. 22 shows stage III and IV lung cancer patients treated with CIT (n=4) versus other treatments (n=4), where the patients had a TF less than 1%. FIG. 23 shows stage III and IV lung cancer patients treated with CIT (n=9) versus other treatments (n=14), where the patients had a TF greater than or equal to 1%. As shown in FIGS. 22-23, patients treated with CIT generally had greater survival probability over a period of time than those treated with other treatments. The difference in benefit is more pronounced in FIG. 23 for patients with higher TF (TF greater than or equal to 1%).

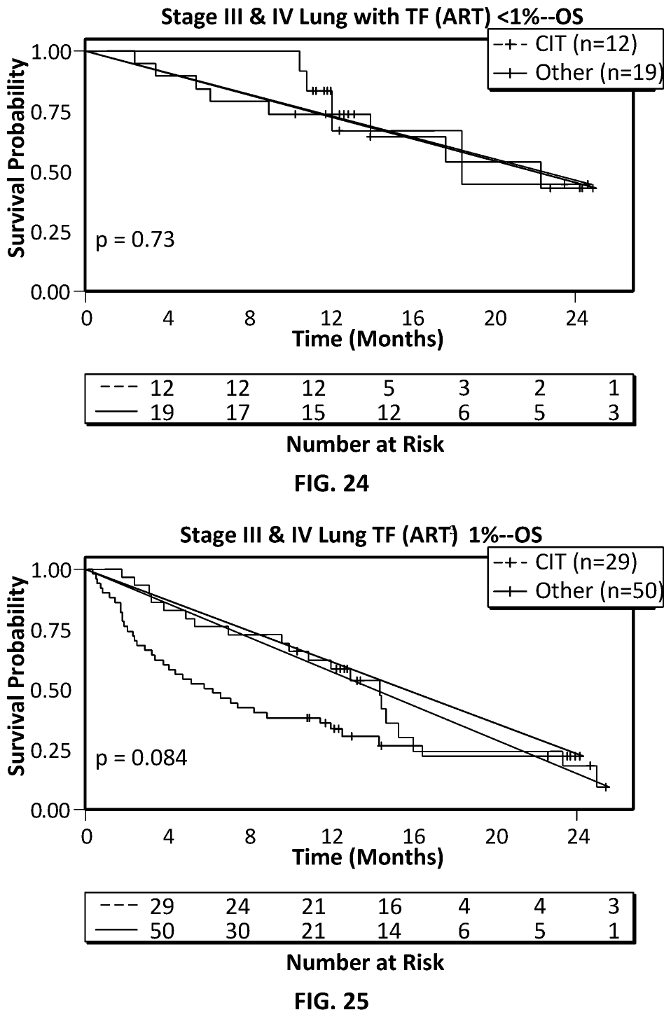

Similarly, FIGS. 24-25 show data demonstrating the use of an estimated TF as a biomarker for CIT response for stage III and IV lung cancer patients. Here, the TF is estimated from ART data gathered from the ART assay, and refers to the max AF of all mutations in the cfDNA. FIG. 24 shows stage III and IV lung cancer patients treated with CIT (n=12) versus other treatments (n=19), where the patients had an ART estimated TF of less than 1%. FIG. 25 shows stage III and IV lung cancer patients treated with CIT (n=29) versus other treatments (n=50), where the patients had an ART estimated TF greater than or equal to 1%. As shown in FIGS. 24-25, patients treated with CIT generally had greater survival probability over a period of time than those treated with other treatments, especially over the first 16 month period. The difference in benefit is more pronounced in FIG. 25 for patients with higher estimated TF (TF greater than or equal to 1%).

Example Computer System

Any of the methods disclosed herein can be performed and/or controlled by one or more computer systems. In some examples, any step of the methods disclosed herein can be wholly, individually, or sequentially performed and/or controlled by one or more computer systems. Any of the computer systems mentioned herein can utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems can be interconnected via a system bus. Additional subsystems include a printer, keyboard, storage device(s), and monitor that is coupled to display adapter. Peripherals and input/output (I/O) devices, which couple to I/O controller, can be connected to the computer system by any number of connections known in the art such as an input/output (I/O) port (e.g., USB, FireWire®). For example, an I/O port or external interface (e.g., Ethernet, Wi-Fi, etc.) can be used to connect a computer system to a wide area network such as the Internet, a mouse input device, or a scanner. The interconnection via system bus allows the central processor to communicate with each subsystem and to control the execution of a plurality of instructions from system memory or the storage device(s) (e.g., a fixed disk, such as a hard drive, or optical disk), as well as the exchange of information between subsystems. The system memory and/or the storage device(s) can embody a computer readable medium. Another subsystem is a data collection device, such as a camera, microphone, accelerometer, and the like. Any of the data mentioned herein can be output from one component to another component and can be output to the user.

A computer system can include a plurality of the same components or subsystems, e.g., connected together by external interface or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

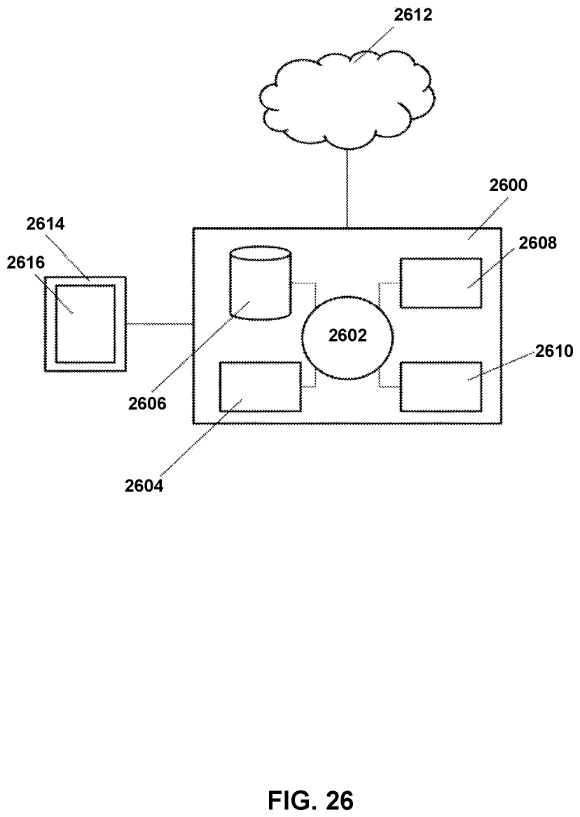

The present disclosure provides computer control systems that are programmed to implement methods of the disclosure for predicting and monitoring treatment response from cell-free nucleic acids. FIG. 26 shows a computer system 2600 that is programmed or otherwise configured to analyze cell-free nucleic acid molecules or sequence reads thereof and determine whether a subject is likely to respond to a treatment in accordance with various embodiments as described herein. The computer system 2600 can implement and/or regulate various aspects of the methods provided in the present disclosure, such as, for example, controlling sequencing of the nucleic acid molecules from a biological sample, performing various steps of the bioinformatics analyses of sequencing data as described herein, integrating data collection, analysis and result reporting, and data management. The computer system 2600 can be an electronic device of a user or a computer system that is remotely located with respect to the electronic device. The electronic device can be a mobile electronic device.

The computer system 2600 includes a central processing unit (CPU, also “processor” and “computer processor” herein) 2602, which can be a single core or multi core processor, or a plurality of processors for parallel processing. The computer system 2600 also includes memory or memory location 2604 (e.g., random-access memory, read-only memory, flash memory), electronic storage unit 2606 (e.g., hard disk), communication interface 2608 (e.g., network adapter) for communicating with one or more other systems, and peripheral devices 2610, such as cache, other memory, data storage and/or electronic display adapters. The memory 2604, storage unit 2606, interface 2608 and peripheral devices 2610 are in communication with the CPU 2602 through a communication bus (solid lines), such as a motherboard. The storage unit 2606 can be a data storage unit (or data repository) for storing data. The computer system 2600 can be operatively coupled to a computer network (“network”) 2612 with the aid of the communication interface 2608. The network 2612 can be the Internet, an internet and/or extranet, or an intranet and/or extranet that is in communication with the Internet. The network 2612 in some cases is a telecommunication and/or data network. The network 2612 can include one or more computer servers, which can enable distributed computing, such as cloud computing. The network 2612, in some cases with the aid of the computer system 2600, can implement a peer-to-peer network, which may enable devices coupled to the computer system 2600 to behave as a client or a server.

The CPU 2602 can execute a sequence of machine-readable instructions, which can be embodied in a program or software. The instructions may be stored in a memory location, such as the memory 2604. The instructions can be directed to the CPU 2602, which can subsequently program or otherwise configure the CPU 2602 to implement methods of the present disclosure. Examples of operations performed by the CPU 2602 can include fetch, decode, execute, and writeback.

The CPU 2602 can be part of a circuit, such as an integrated circuit. One or more other components of the system 2600 can be included in the circuit. In some cases, the circuit is an application specific integrated circuit (ASIC).

The storage unit 2606 can store files, such as drivers, libraries and saved programs. The storage unit 2606 can store user data, e.g., user preferences and user programs. The computer system 2600 in some cases can include one or more additional data storage units that are external to the computer system 2600, such as located on a remote server that is in communication with the computer system 2600 through an intranet or the Internet.

The computer system 2600 can communicate with one or more remote computer systems through the network 2612. For instance, the computer system 2600 can communicate with a remote computer system of a user (e.g., a Smart phone installed with application that receives and displays results of sample analysis sent from the computer system 2600). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2600 via the network 2612.

Methods as described herein can be implemented by way of machine (e.g., computer processor) executable code stored on an electronic storage location of the computer system 2600, such as, for example, on the memory 2604 or electronic storage unit 2606. The machine executable or machine readable code can be provided in the form of software. During use, the code can be executed by the processor 2602. In some cases, the code can be retrieved from the storage unit 2606 and stored on the memory 2604 for ready access by the processor 2602. In some situations, the electronic storage unit 2606 can be precluded, and machine-executable instructions are stored on memory 2604.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that include a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2600 can include or be in communication with an electronic display 2612 that includes a user interface (UI) 2618 for providing, for example, results of sample analysis, such as, but not limited to graphic showings TMB, TH, and/or TF levels in the sample(s), likelihood of response to treatment, and treatment suggestion or recommendation of treatment steps based on the determined TMB, TH, and/or TF as described herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2602. The algorithm can, for example, control sequencing of the nucleic acid molecules from a sample, direct collection of sequencing data, analyzing the sequencing data, performing block-based variant pattern analysis, evaluating the risk, or generating the report indicative of the risk.

In some cases, a sample may be obtained from a subject, such as a human subject. A sample may be subjected to one or more methods as described herein, such as performing an assay. In some cases, an assay may include hybridization, amplification, sequencing, labeling, or any combination thereof. One or more results from a method may be input into a processor 2602. One or more input parameters such as a sample identification, subject identification, sample type, a reference, or other information may be input into a processor 2602. One or more metrics from an assay may be input into a processor 2602 such that the processor may produce a result, such as a classification of pathology (e.g., diagnosis), treatment response likelihood, or a recommendation for a treatment. A processor 2602 may send a result, an input parameter, a metric, a reference, or any combination thereof to a display 2612, such as a visual display or graphical user interface. A processor 2602 may (i) send a result, an input parameter, a metric, or any combination thereof to a server via network 2612, (ii) receive a result, an input parameter, a metric, or any combination thereof from a server via network 2612, (iii) or a combination thereof.

Aspects of the present disclosure can be implemented in the form of control logic using hardware (e.g., an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments described herein using hardware and a combination of hardware and software.

Any of the software components or functions described in this application can be implemented as software code to be executed by a processor using any suitable computer language such as, for example, Java, C, C++, C#, Objective-C, Swift, or scripting language such as Perl or Python using, for example, conventional or object-oriented techniques. The software code can be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium can be any combination of such storage or transmission devices.

Such programs can also be encoded and transmitted using carrier signals adapted for transmission via wired, optical, and/or wireless networks conforming to a variety of protocols, including the Internet. As such, a computer readable medium can be created using a data signal encoded with such programs. Computer readable media encoded with the program code can be packaged with a compatible device or provided separately from other devices (e.g., via Internet download). Any such computer readable medium can reside on or within a single computer product (e.g., a hard drive, a CD, or an entire computer system), and can be present on or within different computer products within a system or network. A computer system can include a monitor, printer, or other suitable display for providing any of the results mentioned herein to a user.

Any of the methods described herein can be totally or partially performed with a computer system including one or more processors, which can be configured to perform the steps. Thus, embodiments can be directed to computer systems configured to perform the steps of any of the methods described herein, with different components performing a respective steps or a respective group of steps. Although presented as numbered steps, steps of methods herein can be performed at a same time or in a different order. Additionally, portions of these steps can be used with portions of other steps from other methods. Also, all or portions of a step can be optional. Additionally, any of the steps of any of the methods can be performed with modules, units, circuits, or other approaches for performing these steps.

### OTHER EMBODIMENTS

The section headings used herein are for organizational purposes only and are not to be construed as limiting the subject matter described.

It is to be understood that the methods described herein are not limited to the particular methodology, protocols, subjects, and sequencing techniques described herein and as such can vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

What is claimed is:

1. A method for determining a subject's likelihood of responding to a treatment by assessing a cell-free DNA (cfDNA) sample collected from the subject, the method comprising:
receiving sequence data gathered from sequencing the cfDNA sample;
generating a feature matrix comprising feature values corresponding to synonymous and nonsynonymous mutations in the sequence data;
predicting a tumor mutational burden (TMB) for a tissue of interest at the subject using a TMB prediction model that receives the feature matrix as input and outputs a predicted TMB;
subsequent to determining the predicted TMB, determining whether a set of criteria has been met, wherein the set of criteria includes at least one criterion that is met when the predicted TMB is high;
in accordance with a determination that the set of criteria has been met, determining that the subject is likely to respond to the treatment; and
in accordance with a determination that the set of criteria has not been met, determining that the subject is not likely to respond to the treatment.

2. The method of claim 1, wherein the predicted TMB is determined to be high when the predicted TMB exceeds a predetermined value.

3. The method of any of claims 1-2, wherein the feature values comprise one or more of:
a number of nonsynonymous somatic mutations for each region of a plurality of regions included in an assay used to sequence the cfDNA sample,
a total number of somatic mutations in the cfDNA sample, and
a total number of nonsynonymous somatic mutations in the cfDNA sample.

4. The method of claim 3, wherein the assay comprises a plurality of regions and each region comprises an individual gene.

5. The method of any of claims 1-4, wherein the predicted TMB represents an estimated total number of nonsynonymous somatic mutations for the tissue of interest at the subject.

6. The method of any of claims 1-5, wherein the treatment comprises an immunotherapy treatment.

7. The method of claim 6, wherein the immunotherapy treatment comprises an immuno oncology treatment.

8. The method of any of claims 1-7, further comprising:
in accordance with the determination that the subject is likely to respond to the treatment, continuing administration of the treatment to the subject; and
in accordance with the determination that the subject is not likely to respond to the treatment, altering administration of the treatment to the subject.

9. The method of any of claims 1-8, wherein the TMB prediction model comprises a statistical model trained with a training set comprising training data obtained from sequencing a plurality of train samples of cfDNA collected from a plurality of subjects, wherein the training data obtained from each train sample corresponds to matched tissue data obtained from a tumoral tissue sample collected from the same subject.

10. The method of any of claim 9, wherein the training data is obtained from targeted sequencing of the plurality of train samples.

11. The method of any of claims 9-10, wherein the matched tissue data is obtained from whole exome sequencing of the tumoral tissue sample.

12. The method of any of claims 9-11, further comprising:
for each train sample in the plurality of train samples:
labeling the training data with a corresponding ground truth TMB determined from the corresponding matched tissue data;
generating a predicted TMB from the labeled training data using the statistical model; and
correlating the predicted TMB with the corresponding ground truth TMB.

13. The method of any of claims 9-12, wherein the statistical model comprises a L1 penalized linear regression model.

14. The method of any of claims 9-13, wherein each train sample corresponds to a cancer stage III or stage IV condition.

15. The method of any of claims 9-14, wherein each train sample of cfDNA has a tumor fraction that exceeds a minimum tumour fraction.

16. The method of claim 15, wherein the tumor fraction comprises a maximum allele frequency of all mutations in the train sample.

17. The method of any of claims 1-16, wherein the set of criteria further includes a criterion that is met when the predicted TMB is high and corresponds to a predicted tumoral heterogeneity (TH) that is indicative of a homogeneous tissue.

18. The method of claim 17, further comprising:
subsequent to the determination that the predicted TMB is high, predicting, based on the sequence data, the TH for the tissue of interest at the subject;
determining whether the predicted TH is indicative of homogeneous or heterogeneous tissue;
in accordance with a determination that the predicted TH is indicative of the homogeneous tissue, determining that the subject is likely to respond to the treatment; and
in accordance with a determination that the predicted TH is indicative of the heterogeneous tissue, determining that the subject is not likely to respond to the treatment.

19. The method of any of claims 17-18, further comprising:
determining the predicted TH using a TH prediction model that receives a set of features in the sequence data as input and outputs the predicted TH, the set of features comprising at least one feature corresponding to one or more of:
an allele frequency of single nucleotide variant (SNV) calls in the cfDNA sample,
a mean allele frequency of cfDNA variants in the cfDNA sample,
a ratio of minimum to maximum allele frequency of cfDNA variants in the cfDNA sample, and
a reciprocal fraction of a number of cfDNA variants in the cfDNA sample.

20. The method of claim 19, wherein the TH prediction model comprises a linear regression model, the method further comprising:
determining, with the TH prediction model, a coefficient of variation of the allele frequency of SNV calls based on the set of features;
in accordance with a determination that the coefficient of variation is low, determining that the predicted TH is indicative of homogeneous tissue; and
in accordance with a determination that the coefficient of variation is high, determining that the predicted TH is indicative of heterogeneous tissue.

21. The method of any of claims 19-20, wherein the TH prediction model comprises a statistical model trained on a training set comprising a plurality of training samples that are derived from ctDNA samples having matched tissue data from tumoral tissue samples, wherein:
training samples having high cfDNA-tissue concordance correspond to low coefficient of variation of cfDNA variant allele frequencies and are homogeneous, and
training samples having low cfDNA-tissue concordance correspond to high coefficient of variation of cfDNA variant allele frequencies and are heterogeneous.

22. The method of any of claims 1-21, wherein the set of criteria further includes a criterion that is met when the predicted TMB is high and a tumor fraction (TF) computed based on the sequence data is low.

23. The method of claim 22, further comprising:
subsequent to the determination that the predicted TMB is high, determining whether the TF is low, wherein the tumor fraction comprises a fraction of tumor-derived cfDNA over a total amount of cfDNA in the cfDNA sample;
in accordance with a determination that the TF is low, determining that the subject is likely to respond to the treatment; and
in accordance with a determination that the TF is not low, determining that the subject is not likely to respond to the treatment.

24. The method of any of claims 1-23, further wherein the cfDNA sample is a blood-based sample.

25. A non-transitory computer-readable medium storing one or more programs, the one or more programs including instructions which, when executed by an electronic device including a processor, cause the device to perform any of the methods of the preceding claims.

26. An electronic device, comprising:
one or more processors;
memory; and
one or more programs, wherein the one or more programs are stored in the memory and configured to be executed by the one or more processors, the one or more programs including instructions for performing any of the methods of the preceding claims.
